(19) United States
(12) Patent Application Publication
(10) Pub. No.: US 2016/0106677 A1
(43) Pub. Date: Apr. 21, 2016

(54) PHARMACEUTICAL COMPOSITION AND USES THEREOF

(71) Applicant: Boehringer Ingelheim International GmbH, Ingelheim am Rhein (DE)

(72) Inventors: Georg BOECK, Laupheim (DE); Venkata VOLETI, Mason, OH (US); Tracy WILLIAMSON, Cincinnati, OH (US)

(21) Appl. No.: 14/884,803

(22) Filed: Oct. 16, 2015

Related U.S. Application Data
(60) Provisional application No. 62/065,161, filed on Oct. 17, 2014.

(51) Int. Cl. A61K 9/24; A61K 31/522; A61K 9/28; A61K 31/155

(52) U.S. Cl. CPC A61K 9/209; A61K 31/155; A61K 31/522; A61K 9/28; A61K 9/282; A61K 9/2866; A61K 9/2853; A61K 9/2813

(57) ABSTRACT

The present invention relates to pharmaceutical compositions comprising fixed dose combinations of a DPP-4 inhibitor drug and metformin XR (extended release), processes for the preparation thereof, and their use to treat certain diseases.

### FIELD OF THE INVENTION

The present invention relates to pharmaceutical compositions containing a fixed dose combination (FDC) comprising a DPP-4 inhibitor drug (particularly 1-[(4-methyl-quinazolin-2-yl)methyl]-3-methyl-7-(2-butyn-1-yl)-8-(3-(R)-amino-piperidin-1-yl)-xanthine, also named linagliptin),

and metformin (particularly metformin hydrochloride) in extended release form (metformin XR); processes for the preparation thereof, and their use to treat certain diseases.

### SUMMARY OF THE INVENTION

In particular, the present invention relates to a pharmaceutical composition comprising

a) an extended release core comprising metformin (particularly metformin hydrochloride) and one or more excipients;

b) a barrier coating; and

c) an immediate release coating comprising a DPP-4 inhibitor, preferably linagliptin, and one or more excipients;

wherein the barrier coating comprises or consists essentially of:

one or more film-coating agents,

an optional plasticizer, and

optionally, a glidant, and/or an anti-tacking agent, and/or a pigment,

wherein the barrier coating is devoid of polydextrose.

Particularly, the present invention relates to a pharmaceutical composition comprising a fixed dose combination of an extended release form of metformin hydrochloride, optionally seal or barrier coated (preferably barrier coated), which is further coated with an immediate release form of 1-[(4-methyl-quinazolin-2-yl)methyl]-3-methyl-7-(2-butyn-1-yl)-8-(3-(R)-amino-piperidin-1-yl)-xanthine (linagliptin).

Further, the present invention relates to a pharmaceutical composition, particularly a solid preparation (e.g. an oral solid dosage form, such as e.g. a tablet), comprising or consisting essentially of
a) an inner extended release core comprising metformin (particularly metformin hydrochloride) and one or more excipients;
b) an optional intermediate seal and/or barrier coating (preferably a barrier coating); and
c) an outer immediate release coating comprising at least one active pharmaceutical ingredient selected from
   a DPP-4 inhibitor, preferably linagliptin,
   and one or more excipients.

In an aspect, the present invention relates to a pharmaceutical composition, particularly a solid preparation (e.g. an oral solid dosage form, such as a tablet) of a selected dipeptidyl peptidase-4 (DPP-4) inhibitor (preferably linagliptin, particularly in immediate release form) and metformin (particularly metformin hydrochloride) in extended release form (metformin XR).

In one embodiment of this aspect, the present invention relates to a pharmaceutical composition, particularly a solid preparation (e.g. an oral solid dosage form, such as a tablet), comprising a fixed dose combination of an extended release form of metformin hydrochloride, optionally seal and/or barrier coated (preferably barrier coated), and further coated with an immediate release form of linagliptin.

In a further aspect, the present invention relates to a pharmaceutical composition, particularly a solid preparation (e.g. an oral solid dosage form, such as e.g. a tablet), comprising

a first component, part or composition comprising metformin (particularly metformin hydrochloride) in extended release form and one or more excipients, and

a second component, part or composition comprising a selected dipeptidyl peptidase-4 (DPP-4) inhibitor (preferably linagliptin), particularly in immediate release form, and one or more excipients.

Particularly, the present invention relates to a pharmaceutical composition, particularly a solid preparation (e.g. an oral solid dosage form, such as a tablet), comprising an extended release form of metformin hydrochloride, optionally seal and/or barrier coated (preferably barrier coated), and further coated with an immediate release form of linagliptin.

In a yet further aspect, the present invention relates to a pharmaceutical composition, particularly a solid preparation (e.g. an oral solid dosage form, such as e.g. a tablet), comprising or consisting essentially of
a) an inner extended release core comprising metformin (particularly metformin hydrochloride) and one or more excipients,
b) a barrier coating, and
c) an immediate release coating comprising a selected dipeptidyl peptidase-4 (DPP-4) inhibitor (preferably linagliptin) and one or more excipients.

Particularly, the pharmaceutical compositions of this invention comprise an inner core formulation of metformin hydrochloride comprising a swellable and/or extended release material.

### DETAILED DESCRIPTION OF THE INVENTION

In one embodiment, the pharmaceutical compositions of this invention comprise an inner extended release core which is a formulation (e.g. matrix formulation) comprising metformin hydrochloride, a swellable and/or extended release material, and one or more further excipients.

Particularly, the pharmaceutical compositions of this invention comprise an outer coat of active pharmaceutical ingredient (API) (linagliptin) in an immediate release polymer film.

The extended-release mechanism for the metformin HCl core may be based on an expandable polymeric swelling formulation that increases gastric retention and extends drug release from the matrix.

This metformin HCl core may be then film-coated (spray-coated) with up to four layers (e.g. barrier coat layer, immediate-release active coat layer, color coat layer, final coat layer), one of which (active coat layer) contains the active pharmaceutical ingredient (API) (linagliptin), to add the immediate-release active component.

Further, the present invention relates to a coating process (e.g. coating technology and processing conditions) and immediate release coating formulations of active pharmaceutical ingredients (API) in low doses (typically in doses of 0.5 to 10 mg or up to 25 mg) on top of tablet cores comprising active pharmaceutical ingredients (API) in high doses (typically in doses of 500-1500 mg) preferably, but not exclusively on extended release tablets. Anyhow, essential parts of the formulation and the process of this invention may be also applicable to any other fixed dose combination with the described setting.

An aim of the present invention is to provide a pharmaceutical composition comprising a combination of a selected DPP-4 inhibitor (preferably linagliptin, particularly in immediate release form), and metformin (particularly metformin hydrochloride) in extended release form.

The objectives of the invention are to identify suitable formulations and processing conditions, such as e.g. of a coat of linagliptin on top metformin XR cores, providing adequate:
Chemical stability of the API (particularly linagliptin) in the API film coat,
Assay of linagliptin in the API film-coat (e.g. 95-105%),
Content uniformity of linagliptin (e.g. RSD <3%) in the API film-coat,
Low defect rate of the API-film during film coating process,
Fast dissolution of the API from the API film-coat and no changes of XR Metformin HCl dissolution, due to the API coating with immediate release of linagliptin,
Processing aspects of coating process/technology, processing conditions and immediate release API (linagliptin) coating formulations (API film coat),
Processing aspects of coating process/technology, processing conditions and immediate release API (linagliptin) coating formulations on top of metformin extended release tablets.

A particular objective of the present invention is to provide a pharmaceutical composition and suitable coating process with very broad range of drug substance (linagliptin)/drug substance (metformin) ratio: 1:400-1:40. And the ratio of very low dosed API, e.g. linagliptin with 1 mg or 2.5 mg or 5 mg to very high dosed metformin with 1000 mg and more. And the suitable immediate release dissolution of the low dosed API with high dosed extended release metformin.

A more particular aim of the present invention is to provide an optimized pharmaceutical composition such as comprising an optimized barrier coating improved for stability of the composition.

The unit dosage strength of the metformin hydrochloride for incorporation into the fixed-dose combination of the present invention is preferably 500, 750, 850 or 1000 milligrams, or even more (e.g. 1500 mg).

These unit dosage strengths of metformin hydrochloride represent the dosage strengths approved in the U.S. for marketing to treat Type 2 diabetes.

The unit dosage strength of linagliptin for incorporation into the fixed-dose combination of the present invention is preferably in the range from 0.5 to 10 mg, for particular example 2.5 or 5 milligrams, or even less (e.g. 0.5 mg or 1 mg).

Specific embodiments of dosage strengths for linagliptin and metformin hydrochloride in the fixed-dose combinations of the present invention are the following:

(1) 5 milligrams of linagliptin and 1000 milligrams metformin hydrochloride;

(2) 2.5 milligrams of linagliptin and 1000 milligrams metformin hydrochloride;

(3) 2.5 milligrams of linagliptin and 750 milligrams metformin hydrochloride.

Optionally, the pharmaceutical compositions, particularly solid preparations (e.g. oral solid dosage forms, such as e.g. tablets) according to the present invention may further comprise a color coat and/or a final coat.

It has been found that absence or replacement of polydextrose in the barrier coat according to the present invention improves stability of the composition and provides good coating performance.

(a) Metformin Part:

The first part in the present invention is a part (composition, particularly solid composition, e.g. a solid pharmaceutical composition for oral administration, e.g. tablet) comprising metformin (particularly metformin hydrochloride) in extended release form, particularly an extended release formulation of metformin.

In an embodiment, this first part is a metformin (e.g. 500 mg, 750 mg or 1000 mg metformin hydrochloride) extended release core.

Exemplary extended release formulations of metformin are disclosed in U.S. Pat. No. 6,340,475; U.S. Pat. No. 6,488,962; U.S. Pat. No. 6,635,280; U.S. Pat. No. 6,723,340; U.S. Pat. No. 7,780,987; U.S. Pat. No. 6,866,866; U.S. Pat. No. 6,495,162; U.S. Pat. No. 6,790,459; U.S. Pat. No. 6,866,866; U.S. Pat. No. 6,475,521; and U.S. Pat. No. 6,660,300; the disclosures of which are incorporated herein in their entireties.

A particular extended release formulation of metformin is described in U.S. Pat. No. 6,340,475 or U.S. Pat. No. 6,723,340, the disclosure of which is incorporated herein in its entirety.

In an embodiment, the fixed-dose combination products of the present invention comprise—as first part—an inner core matrix formulation with metformin hydrochloride dispersed therein, said matrix formulation containing an extended release material. The matrix formulation is compressed into a tablet form.

In a particular embodiment, the fixed-dose combination products of the present invention comprise—as first part—an inner core extended release formulation comprising metformin hydrochloride, hydroxypropyl methylcellulose (hypromellose), polyethylene oxide, microcrystalline cellulose (which may be optional), and magnesium stearate.

In another particular embodiment, the fixed-dose combination products of the present invention comprise—as first part—an inner core extended release formulation comprising metformin hydrochloride, polyethylene oxide, low molecular weight hydroxypropyl methylcellulose (hypromellose, e.g. Methocel™ E5, available from Dow Chemical Co., Midland, Mich.), and magnesium stearate.

A particular extended release formulation of metformin is described in U.S. Pat. No. 6,723,340 as follows:

In an embodiment, the extended release material of the matrix comprises poly(ethylene oxide) and/or hydroxypropyl methylcellulose (HPMC), such as e.g. a combination of poly(ethylene oxide) and hydroxypropyl methylcellulose (HPMC), preferably at a weight ratio that causes the matrix to swell upon contact with gastric fluid to a size large enough to provide gastric retention.

In a particular embodiment, the extended release material of the matrix comprises or consists essentially of poly(ethylene oxide).

The poly(ethylene oxide) component of the matrix may limit initial release of the drug and may impart gastric retention through swelling. The hydroxypropyl methylcellulose (HPMC) component may lower the amount of poly(ethylene oxide) required while still allowing the swelling to occur.

Preferably, the poly(ethylene oxide) has a viscosity average molecular weight of from about 2,000,000 to about 10,000,000 daltons, more preferably from about 4,000,000 to about 7,000,000 daltons.

Preferably, the hydroxypropyl methylcellulose (HPMC) has a viscosity of from about 4,000 centipoise to about 200,000 centipoise, more preferably from about 50,000 to about 200,000 centipoise, even more preferably 80,000 centipoise to about 120,000 centipoise, measured as a 2% solution in water.

More preferably, the poly(ethylene oxide) has a viscosity average molecular weight of from about 4,000,000 to about 7,000,000 daltons, and the hydroxypropyl methylcellulose (HPMC) has a viscosity of from about 80,000 centipoise to about 120,000 centipoise, measured as a 2% solution in water.

In an embodiment, the weight ratio of the poly(ethylene oxide) to hydroxypropyl methylcellulose (HPMC) is within the range from about 1:3 to about 3:1, such as e.g. 1:2 to 2:1.

In a preferred embodiment, the HPMC (HPMC having a viscosity of from about 4,000 centipoise to about 200,000 centipoise, more preferably from about 50,000 to about 200,000 centipoise, even more preferably 80,000 centipoise to about 120,000 centipoise, measured as a 2% solution in water, such as e.g. Methocel™ K100M) in the matrix is optional or is absent.

In a further embodiment, the weight ratio of the poly(ethylene oxide) and, optionally, hydroxypropyl methylcellulose (HPMC) in combination constitutes from about 15% to about 90%, or from about 30% to about 65%, or from about 40% to about 50%, by weight of the metformin part.

Tablet cores in accordance with this invention can be prepared by common tabletting methods that involve mixing, comminution, and fabrication steps commonly practiced by and well known to those skilled in the art of manufacturing drug formulations. Examples of such techniques are:

(1) Direct compression using appropriate punches and dies, typically fitted to a suitable rotary tabletting press;

(2) Injection or compression molding;

(3) Granulation by fluid bed, by low or high shear granulation, or by roller compaction, followed by compression; and

(4) Extrusion of a paste into a mold or to an extrudate to be cut into lengths.

When tablets are made by direct compression, the addition of lubricants may be helpful and is sometimes important to promote powder flow and to prevent breaking of the tablet when the pressure is relieved. Examples of typical lubricants are magnesium stearate (in a concentration of from 0.25% to 3% by weight, preferably about 1% or less by weight, in the powder mix), stearic acid (0.5% to 3% by weight), and hydrogenated vegetable oil (preferably hydrogenated and refined triglycerides of stearic and palmitic acids at about 1% to 5% by weight, most preferably about 2% by weight).

Additional excipients may be added, such as e.g. granulating aids (e.g. low molecular weight HPMC at 2-5% by weight), binders (e.g. microcrystalline cellulose, or low molecular weight HPMC), and additives to enhance powder flowability, tablet hardness, and tablet friability and to reduce adherence to the die wall.

An extended release metformin tablet core comprises metformin hydrochloride, a combination of poly(ethylene oxide) and hydroxypropyl methylcellulose (e.g. Methocel® K100M) (which may be optional) as a matrix for a swellable extended release tablet, microcrystalline cellulose as binder (which may be optional), low molecular weight hydroxypropyl methylcellulose (e.g. Methocel™ E5) as granulating aid or as binder, and magnesium stearate as lubricant.

The composition of an embodimental metformin core tablet is provided as follows: metformin hydrochloride, e.g. 49.97% by weight of the first part, poly(ethylene oxide), e.g. 26.50% by weight of the first part, hydroxypropyl methylcellulose (e.g. Methocel™ K100M), e.g. 16.08% by weight of the first part,

microcrystalline cellulose, e.g. 4.99% by weight of the first part,

low molecular weight hydroxypropyl methylcellulose (e.g. Methocel™ E5), e.g. 1.70% by weight of the first part, and

magnesium stearate, e.g. 0.75% by weight of the first part.

Tablets may be formulated by dry blending a granulation comprising metformin hydrochloride and low molecular weight HPMC (e.g. Methocel™ E5) and the remaining excipients listed above, followed by pressing on a tablet press.

Such an extended release matrix formulation of metformin is disclosed in U.S. Pat. No. 6,723,340 (e.g. Example 3), the disclosure of which is incorporated herein in its entirety.

As further example of a lubricant sodium stearyl fumarate may be mentioned (e.g. at about 0.25-3% by weight).

Preferably, another particular extended release formulation of metformin comprises or consists essentially of metformin hydrochloride (e.g. 750 mg or 1000 mg), poly(ethylene oxide) (e.g. SENTRY™ POLYOX™ WSR 303) as swelling and/or extended release polymer, low molecular weight hydroxypropyl methylcellulose (hypromellose, e.g. Methocel™ E5) as binder, and magnesium stearate as lubricant.

In a certain embodiment, the poly(ethylene oxide) as swelling and/or extended release polymer has an approximate molecular weight of 7,000,000, and/or a viscosity range 7500-10,000 centipoise measured as aqueous 1% solution at 25° C.

In a preferred embodiment, the poly(ethylene oxide) is SENTRY™ POLYOX™ WSR 303.

In a certain embodiment, the low molecular weight hydroxypropyl methylcellulose as (granulation) binder is hypromellose having viscosity of 5 centipoise measured as aqueous 2% solution at 20° C.

In a preferred embodiment, the low molecular weight hydroxypropyl methylcellulose is Methocel™ E5.

In a further embodiment, the weight ratio of the poly(ethylene oxide) to hydroxypropyl methylcellulose (low molecular weight HPMC) is within the range from about 10:1 to 20:1, preferably about 10:1 to about 15:1.

In another further embodiment, the weight ratio of the poly(ethylene oxide) and, optionally, hydroxypropyl methylcellulose (HPMC) in combination constitutes from about 15% to about 50%, or from about 20% to about 40%, or from about 25% to about 35%, by weight of the metformin part.

Preferably, the composition of a representative embodimental metformin extended release core is provided as follows:

metformin hydrochloride, e.g. 750.0 mg, 65.68% by weight of the first part, poly(ethylene oxide) (SENTRY™ POLYOX™ WSR 303), e.g. 355.2 mg, 31.10% by weight of the first part,

low molecular weight hydroxypropyl methylcellulose (Methocel™ E5), e.g. 25.5 mg, 2.23% by weight of the first part, and magnesium stearate, e.g. 11.3 mg, 0.99% by weight of the first part.

The composition of another representative embodimental metformin extended release core is provided as follows:

metformin hydrochloride, e.g. 1000.0 mg, 70.47% by weight of the first part, poly(ethylene oxide) (SENTRY™ POLYOX™ WSR 303), e.g. 370.0 mg, 26.07% by weight of the first part,

low molecular weight hydroxypropyl methylcellulose (Methocel™ E5), e.g. 34.0 mg, 2.40% by weight of the first part, and

magnesium stearate, e.g. 15.0 mg, 1.06% by weight of the first part.

The manufacturing of metformin (e.g. 750 mg or 1000 mg metformin hydrochloride) extended release cores comprises metformin wet granulation (e.g. high shear granulation) in the presence of low molecular weight HPMC (hypromellose, Methocel™ E5) as binder for granulation, followed by optional wet milling, drying (e.g. fluid bed drying, e.g. until moisture content (as determined by loss on drying) is not more than 2%) and milling (e.g. dry granulation milling). The milled granulation is subsequently used for blending with poly(ethylene oxide) (SENTRY™ POLYOX™ WSR 303), then magnesium stearate as lubricant for tableting is added for final blending. The obtained metformin blend is used for compression of single layer tablets.

In the metformin granulation process the metformin hydrochloride (750 mg or 1000 mg, 96.71% by weight of total dry granulate) and part of hypromellose (Methocel™ E5, e.g. 2.39% by weight of total dry granulate) are premixed and granulated with an aqueous (10% w/w) binder solution of remaining part of hypromellose (Methocel™ E5, e.g. 0.90% by weight of total dry granulate) using a high shear granulator.

Before being used in the manufacturing process, the excipients during dispensing may be pre-sieved and the metformin HCl may be pre-milled.

In a further embodiment, the metformin extended release formulation allows for targeted, controlled delivery of metformin to the upper gastrointestinal (GI) tract. In a further embodiment, the metformin extended release formulation is a hydrogel matrix system and contains a swelling hydrophilic polymer and further excipients, which may allow the metformin tablet core to be retained in the stomach (‘gastric retention’) for approximately eight to nine hours. During this time, the tablet core's metformin is steadily delivered to the upper GI tract at the desired rate and time, without potentially irritating ‘burst’ of drug. This gradual, extended release typically allows for more of the metformin drug to be absorbed in the upper GI tract and minimizes the amount of drug that passes through to the lower GI tract.

In another embodiment, the metformin XR (e.g. 750 mg or 1000 mg) cores of this invention provide gastric retention with extended dose of metformin released over approximately 12 hours.

(b1) Linagliptin Part:

In one variant, the second part in the present invention is a part (composition, particularly film coat) comprising linagliptin in immediate release form.

In an embodiment, the fixed-dose combination products of the present invention comprise—as second part—a film coat formulation of linagliptin, said film coat formulation comprising linagliptin, a stabilizer for stabilizing linagliptin (e.g. a basic and/or nucleophilic excipient, preferably L-arginine as stabilizer), a film-coating agent (such as e.g. hydroxypropyl methylcellulose, e.g. Hypromellose 2910, Methocel™ E5, or Methocel™ E15), a plasticizer (such as e.g. polyethylene glycol, e.g. Macrogol 400, 6000 or 8000, or propylene glycol), and, optionally, a glidant and/or anti-tacking agent (such as e.g. talc).

In an embodiment, the weight ratio of the L-arginine to linagliptin is within the range from about 20:1 to about 1:10, or from about 15:1 to about 1:10, or from about 10:1 to about 1:10, or from about 5:1 to about 1:5, or from about 4:1 to about 1:1, or from about 3:1 to about 1:1, or from about 4:1 to about 2:1, especially about 4:1.

In a further embodiment, the weight ratio of the L-arginine to linagliptin is within the range from about 2:1 to about 1:1, up to about 0.2:1.

In a preferred embodiment, the weight ratio of the L-arginine to linagliptin is within the range from about 4:1 to about 2:1, especially about 4:1.

In an embodiment, the linagliptin immediate release coating comprises or consists essentially of:

linagliptin,

L-arginine,

either polyethylene glycol or propylene glycol,

hydroxypropyl methylcellulose, and

optionally talc.

For example, the composition of a representative linagliptin containing film coat is provided as follows:
linagliptin, e.g. 2.5 mg or 5 mg;
L-arginine, e.g. depending from need of stabilizer amount, e.g. in the range from about 0.5 mg to about 10 mg-12.5 mg (e.g. 5 mg or 10 mg), or up to about 20 mg-25 mg (e.g. 20 mg);
hydroxypropyl methylcellulose (e.g. Methocel™ E5, Methocel™ E15, or Pharmacoat® 603 or 606), e.g. from about 25 mg to about 40 mg (especially from 34.5 mg to 38 mg, or 34.5 mg or 38 mg);
polyethylene glycol (e.g. Macrogol 400, 6000 or 8000), e.g. from about 0 to about 12 mg, especially 10-12 mg;
propylene glycol, e.g. from about 0 mg to about 15 mg (especially 9 mg or 0 mg); and
talc, e.g. from about 0 mg to about 15 mg (especially 9 mg).

Depending from need of stabilizer the amount of L-arginine may be in the range from 0.5 mg to 10 mg-12.5 mg, or up to about 20 mg-25 mg. With different dose and different arginine amount, the arginine amount may be substituted by hydroxypropyl methylcellulose (HPMC).

In an embodiment, polyethylene glycol and propylene glycol are mutually exclusive in above composition, i.e. if polyethylene glycol is present then propylene glycol is absent, or if propylene glycol is present then polyethylene glycol is absent.

The composition of a representative linagliptin containing film coat suspension further comprises water, e.g. from about 240 mg to about 1440 mg, especially in the range from 904 mg to 1440 mg. The total solids concentration of the suspension is from about 4% to about 12.5% w/w, especially from 4% to 6% w/w. Viscosity may be from about 10 mPas to 110 mPas (e.g. 46-56 mPas).

The sum solids of the linagliptin coating suspension is from about 50 mg to about 120 mg. For example, the sum solids is 60 mg of solid amount of the film coating suspension for 2.5 mg linagliptin, and 120 mg sum solid amount of the film coating suspension for 5 mg linagliptin. Therefore with the same formulation of linagliptin and double coating time (i.e. double amount of coating suspension) it is possible to prepare the higher dose range of linagliptin. Hence different dosage strengths can be achieved by altering coating (spraying) times.

Further embodiments as to (b1):

L-Arginine is preferably necessary for the stabilization of linagliptin. Alternatively or additionally, a seal coat and/or a barrier coat may be used between the metformin XR core and the linagliptin-containing film coat.

In an embodiment, a seal and/or a barrier coat is present between the metformin XR core and the linagliptin-containing film coat (preferably further containing L-arginine). In another embodiment, the seal and/or barrier coat is absent between the metformin XR core and the linagliptin-containing film coat (preferably further containing L-arginine).

In some embodiments, the barrier coat may be from 0% to 20% w/w or from 1% to 20% w/w or from 3% to 7% w/w (e.g. 0%, 6%, 12% or 18%) per weight of the total linagliptin/metformin HCl containing composition. Preferably, the barrier coat may have a weight in the range from 50 to 100 mg, for example 60-75 mg (such as for the formulation containing 2.5 mg/750 mg linagliptin/metformin HCl) or 75-95 mg (such as for the formulation containing 2.5 mg/1000 mg or 5 mg/1000 mg linagliptin/metformin HCl).

In a further embodiment, a seal coat and/or a barrier coat is/are present between the metformin XR core and the linagliptin-containing film coat (preferably further containing L-arginine).

In a preferred embodiment, a barrier coat is present between the metformin XR core and the linagliptin-containing film coat (preferably further containing L-arginine).

Alternatively or further to the API film coats of (b1), for the API (linagliptin) containing film coat, a film coat comprising a mixture of hydroxypropylcellulose and hydroxypropyl methylcellulose, or a mixture of polyvinyl alcohol (PVA) and polyethylene glycol (PEG); or a commercial film-coat such as Opadry®, Opadry II® or other Opadry IR film coat, which are formulated powder blends provided by Colorcon®, Harleysville, Pa., may be used. With Opadry II® or PVA based API coating higher solid concentrations and shorter coating time durations are possible, therefore it works in a range of 10-30%, especially 20% solid concentration. This higher solid concentration, e.g. 20%, typically results in a shorter coating time, e.g. 2-5 hours.

In further embodiments of the API containing film coating layers of this invention, the film-coating agent may be one or more of hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), polyvinyl alcohol (PVA), ethyl cellulose, hydroxypropyl cellulose, polydextrose, methacrylic and/or acrylic polymer, or mixtures thereof (e.g. a mixture of one or more HPMC and polydextrose).

In further embodiments of the API containing film coating layers of this invention, the plasticizer may be one or more of polyethylene glycol (PEG, such as e.g. Macrogol 400, 3000, 4000, 6000 and/or 8000), propylene glycol, diethyl phthalate, tributyl sebacate and/or triacetin, or mixtures thereof (e.g. a mixture of polyethylene glycol and triacetin).

In a further embodiment of this invention, a API containing film coat according to this invention may comprise or consist essentially of

the API, preferably a stabilizer for stabilizing linagliptin (preferably L-arginine), one or more film-coating agents, e.g. selected from hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), polyvinyl alcohol (PVA), ethyl cellulose, hydroxypropyl cellulose, polydextrose, methacrylic and/or acrylic polymer, or a mixture thereof, optionally one or more plasticizers, e.g. selected from polyethylene glycol (PEG, such as e.g. Macrogol 400, 3000, 4000, 6000 and/or 8000), propylene glycol, diethyl phthalate, tributyl sebacate and/or triacetin, or a mixture thereof,

optionally a glidant, e.g. talc, magnesium stearate or fumed silica, and

optionally one or more pigments (e.g titanium dioxide) and/or colors (e.g. based on iron oxides).

Accordingly, the API containing film coating layers according to this invention may comprise one or more of the following excipients:

at least one film-coating agent, such as e.g. hydroxypropyl methylcellulose (HPMC, hypromellose such as e.g. hypromellose 2910 with nominal viscosity of 3, 5, 6, 15 or 50 cP, Methocel™ E5, Methocel™ E15, Pharmacoat® 603, Pharmacoat® 606, or Pharmacoat® 615), polyvinyl alcohol (PVA), ethyl cellulose, hydroxypropyl cellulose, polydextrose, or a methacrylic or acrylic polymer,

at least one plasticizer, such as e.g. polyethylene glycol (PEG, such as e.g. Macrogol 400, 3000, 4000, 6000 or 8000), propylene glycol, diethyl phthalate, tributyl sebacate or triacetin,

at least one glidant or anti-adherent, such as e.g. talc, magnesium stearate or fumed silica, and

at least one pigment, such as e.g. titanium dioxide, and/or colorant (such as e.g. based on an iron oxide).

In certain embodiments, the hydroxypropyl methylcellulose (HPMC) as film-coating agent is hypromellose having viscosity from about 3 centipoise to about 15 centipoise or up to 40-60 centipoise, measured as aqueous 2% solution at 20° C., e.g. 3, 5, 6, 15 or 50 cP, such as e.g. hypromellose 2910 with nominal viscosity of 3 cP (HPMC 2910-3), hypromellose 2910 with nominal viscosity of 5 cP (HPMC 2910-5), hypromellose 2910 with nominal viscosity of 6 cP (HPMC 2910-6), hypromellose 2910 with nominal viscosity of 15 cP (HPMC 2910-15), hypromellose 2910 with nominal viscosity of 50 cP (HPMC 2910-50), Methocel™ E5, Methocel™ E15, Pharmacoat® 603, Pharmacoat® 606, or Pharmacoat® 615.

In certain embodiments, the polyethylene glycol (PEG) as plasticizer is macrogol having average molecular weight from about 400 to about 8000 daltons, e.g. 400, 1500, 3000, 4000, 6000 or 8000 D, such as e.g. Marcrogol 400, Marcrogol 6000 or Marcrogol 8000.

In certain embodiments, the coating material for API coating is commercially available under the trade name Opadry®, Opadry II® or other Opadry® film coat.

For example, the composition of a representative linagliptin containing film coat may be provided as follows:
linagliptin, such as e.g. from 0.5 mg to 10 mg (e.g. 2.5 mg or 5 mg);
L-arginine, e.g. depending from need of stabilizer amount, e.g. the weight ratio L-arginine:linagliptin may be 1:1 to 5:1, such as e.g. in the range from about 0.5 mg to about 10 mg −12.5 mg (e.g. 5 mg or 10 mg) or up to about 20 mg-25 mg (e.g. 20 mg);
hydroxypropyl methylcellulose (e.g. Methocel™ E5, Methocel™ E15, or Pharmacoat® 603 or 606), e.g. from about 25 mg to about 40 mg (especially from 34.5 mg to 38 mg, or 34.5 mg or 38 mg);
polyethylene glycol (e.g. Macrogol 400, 6000 or 8000), e.g. from about 0 to about 15 mg, especially 10-12 mg;
propylene glycol, e.g. from about 0 mg to about 15 mg (especially 9 mg or 0 mg); and
talc, e.g. from about 0 mg to about 15 mg (especially 9 mg).

For example, an API (linagliptin) containing film coat according to this invention may comprise or consist essentially of
linagliptin, such as e.g. from 0.5 mg to 10 mg (e.g. 2.5 mg or 5 mg),
L-arginine (as stabilizer, e.g. the weight ratio L-arginine:linagliptin may be 1:1 to 5:1, such as e.g. in the range from about 0.5 mg to about 25 mg, preferably from about 5 mg to about 20 mg, more preferably from about 10 mg to about 20 mg),
a film forming agent (e.g. based on HPMC (HPMC 2910-3), e.g. in the range from about 25 mg to about 40 mg, such as a mixture of hypromellose 2910 (HPMC 2910-3, 95% w/w) and PEG (PEG 8000, 5% w/w)),
a plasticizer (e.g. based on PEG (PEG 6000), e.g. in the range from about 1 mg to about 12 mg, especially from about 10 mg to about 12 mg, and
a glidant (e.g. talc, e.g. in the range from about 1 mg to about 15 mg, especially about 9 mg).

For particular example, an immediate release API coating of this invention comprises or consist essentially of:

polyethylene glycol (e.g. polyethylene glycol of molecular weight of about 6000 and/or 8000, e.g. 8-25% w/w, preferably 10-20% w/w, such as 16.8 or 14.3% w/w),

linagliptin (as API, e.g. 1-10% w/w, preferably 2-7% w/w, such as 3.5 or 6.0% w/w), L-arginine (as stabilizer, e.g. the weight ratio L-arginine:linagliptin may be 1:1 to 5:1,

preferably 2:1 to 4:1, especially 4:1; such as e.g. 4-40% w/w, preferably 10-30% w/w, such as 14 or 24% w/w),

hydroxypropyl methylcellulose (e.g. hydroxypropyl methylcellulose of 3 cP, e.g. 40-70% w/w, preferably 40-65% w/w, such as 53.2 or 45.2% w/w), and

talc (e.g. 5-20% w/w, preferably 8-16% w/w, such as 12.5 or 10.7% w/w),

wherein % w/w relate to the weight of the immediate release coating.

For more particular example, an immediate release API coating of this invention comprises: polyethylene glycol of molecular weight of about 6000 (e.g. 8-20% w/w, preferably 10-18 w/w, such as 14.0 or 11.9% w/w),

linagliptin (as API, e.g. 1-10% w/w, preferably 2-7% w/w, such as 3.5 or 6.0% w/w),

L-arginine (as stabilizer, e.g. the weight ratio L-arginine: linagliptin may be 1:1 to 5:1, preferably 2:1 to 4:1, especially 4:1; such as e.g. 4-40% w/w, preferably 10-30% w/w, such as 14 or 24% w/w),

hydroxypropyl methylcellulose (e.g. hydroxypropyl methylcellulose of 3 cP, e.g. 40-70% w/w, preferably 40-65% w/w, such as 53.2 or 45.2% w/w),

polyethylene glycol of molecular weight of about 8000 (e.g. 0-5% w/w, preferably 2-4% w/w, such as 2.8 or 2.4% w/w), and

talc (e.g. 5-20% w/w, preferably 8-16% w/w, such as 12.5 or 10.7% w/w),

wherein % w/w relate to the weight of the immediate release coating, and

wherein the content of linagliptin may be in the range from 1 to 10 mg, particularly 2.5 mg or 5.0 mg.

For further example, further versions of (API-containing) film coat compositions comprising one or more of the following ingredients of Tables 1 or 2, of Tables 3 or 4 or of Tables 5, 6a, 6b, 6c may be provided, e.g. as follows from Tables 1 or 2, or from Tables 3 or 4 or from Tables 5, 6a, 6b, 6c:

TABLE 1
Example (prototypical) formulations for API-coating of linagliptin
on top of metformin XR cores

| | | PEG- | | | | |
|---|---|---|---|---|---|---|
| | | containing | PG- | PG- | | |
| | PEG- | version | containing | containing | Further | Further |
| | containing | (reduced | version | version | version | version |
| | version | arginine) | (low DL) | (high DL) | (e.g. | (e.g. |
| Composition | (e.g. 2.5 | (e.g. 5 mg | (e.g. 2.5 | (e.g. 2.5 | 2.5 mg | 5 mg |
| (% w/w) | mg API) | API) | mg API) | mg API) | API) | API) |
| Linagliptin | 4.20 | 4.39 | 4.55 | 5.29 | 4.16 | 4.16 |
| HPMC (e.g | 67.23 | 70.18 | 72.73 | 70.55 | — | — |
| Pharmacoat ® | | | | | | |
| 615)* | | | | | | |
| HPMC (e.g | — | — | — | — | 57.5 | 57.5 |
| Methocel ™ E5) | | | | | | |
| Polyethylene | 20.17 | 21.05 | — | — | 15 | 15 |
| glycol (e.g. PEG | | | | | | |
| 6000) | | | | | | |
| Propylene glycol | — | — | 3.64 | 3.53 | — | — |
| L-Arginine | 8.40 | 4.39 | 9.09 | 10.58 | 8.33 | 8.33 |
| Talc | — | — | 10.00 | 10.05 | 15 | 15 |
| Purified water | ** | ** | ** | ** | ** | ** |
| Total | 100.00 | 100.00 | 100.00 | 100.00 | 100.00 | 100.00 |
| Solid content of | 5.95 | 5.70 | 5.50 | 5.67 | 4.0 | 4.0 |
| suspension (%) | | | | | | |

*Alternative Methocel ™ E15
**Solvent is a volatile component, which does not remain in the final product

In one embodiment of the API coatings of this invention, the film-coating agent used is highly viscous.

In another embodiment of the API coatings of this invention, the film-coating agent used is low viscous.

TABLE 2
Further Example (prototypical) formulations for API-coating of
linagliptin on top of metformin XR cores:

| | | PEG- | PG- | PG- |
|---|---|---|---|---|
| | PEG- | containing | containing | containing |
| | containing | version | version | version |
| | version | (reduced | (low DL) | (high DL) |
| | (e.g. | arginine) | (e.g. | (e.g. |
| Composition | 2.5 mg | (e.g. 5 mg | 2.5 mg | 2.5 mg |
| (% w/w) | API) | API) | API) | API) |
| Linagliptin | 4.20 | 4.39 | 4.55 | 5.29 |
| HPMC (e.g. | 67.23 | 70.18 | 72.73 | 70.55 |
| Pharmacoat ® 615) | | | | |
| Polyethylene glycol | 20.17 | 21.05 | — | — |
| (e.g. PEG 6000) | | | | |
| Propylene glycol | — | — | 3.64 | 3.53 |
| L-Arginine | 8.40 | 4.39 | 9.09 | 10.58 |
| Talc | — | — | 10.00 | 10.05 |
| Purified water | ** | ** | ** | ** |
| Total | 100.00 | 100.00 | 100.00 | 100.00 |
| Solid content of | 5.95 | 5.70 | 5.50 | 5.67 |
| suspension (%) | | | | |

** Solvent is a volatile component, which does not remain in the final product

TABLE 3
Example formulations for extended release core comprising metformin
hydrochloride, barrier film coating and API-film coating of linagliptin:
Component
Extended release core
Metformin hydrochloride (Metformin HCl)
Polyethylene Oxide
(e.g. high molecular weight, e.g. about 7,000,000, such as
WSR-303)
Hydroxypropyl methylcellulose (Hypromellose)
(e.g. viscosity about 5 mPas for a 2% solution in water at
20° C., such as HPMC 2910-5 (Methocel ™ E5))
Magnesium Stearate
Barrier coat:
Film Forming System:
Hydroxypropyl cellulose (e.g. 33.75% w/w),
Hydroxypropyl methylcellulose (e.g. HPMC of 3 cP, such
as Hypromellose 2910 3 cP, e.g. 33.75% w/w),
Talc (e.g. 20.0% w/w) and
Titanium dioxide (e.g. 12.5% w/w)
Active (API) coat:
Linagliptin (as API)
Film Forming System:
Hydroxypropyl methylcellulose (e.g. HPMC of 3 cP, such
as HPMC 2910-3, e.g. 95% w/w)
Polyethylene glycol (e.g. PEG 8000, e.g. 5% w/w)
L-Arginine
Polyethylene glycol (e.g. PEG 6000)
Talc

TABLE 4
Example formulation for barrier-film coating between
metformin XR cores and API-film coat of linagliptin:

| Composition (% w/w) | Ex. 4.0 |
|---|---|
| Hydroxypropyl cellulose | 33.75 |
| Hydroxypropyl | 33.75 |
| methylcellulose | |
| (e.g. HPMC of 3 cP, | |
| such as HPMC 2910-3) | |
| Talc | 20.0 |
| Titanium dioxide | 12.5 |
| Purified water | * |
| Total | 100.0 |

*Processing agent water used in manufacturing and removed by drying

TABLE 5
Example formulations for API-coating of linagliptin on top of
metformin XR cores and an optional seal or barrier coating layer:

| | Ex. 5.1 | Ex. 5.2 |
|---|---|---|
| | (e.g. 2.5 mg | (e.g. 5 mg |
| Composition (% w/w) | API) | API) |
| Polyethylene glycol | 14.0 | 11.9 |
| (e.g. PEG 6000) | | |
| Linagliptin | 3.5 | 6.0 |
| L-Arginine | 14.0 | 23.8 |
| Hydroxypropyl | 53.2 | 45.2 |
| methylcellulose | | |
| (e.g. HPMC of 3 cP, | | |
| such as HPMC- | | |
| 2910-3) | | |
| Polyethylene glycol | 2.8 | 2.4 |
| (e.g. PEG 8000) | | |
| Talc | 12.5 | 10.7 |
| Purified water | * | * |
| Total | 100.0 | 100.0 |

*Processing agent water used in manufacturing and removed by drying

TABLE 6a
Further Example formulations for extended release core comprising metformin
hydrochloride, barrier coating and API-coating of linagliptin

| Component | mg/tablet |
|---|---|
| Extended release core | |
| Metformin hydrochloride (Metformin HCl) | 750 |
| Polyethylene Oxide | 355.2 |
| (e.g. high molecular weight, e.g. about 7,000,000, such as | |
| WSR-303) | |
| Hydroxypropyl methylcellulose (Hypromellose) | 25.5 |
| (e.g. viscosity about 5 mPas for a 2% solution in water at | |
| 20° C., such as HPMC 2910-5 (Methocel ™ E5)) | |
| Magnesium Stearate | 11.3 |
| Barrier coat: | |
| Film Forming System: | 68.2 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 2.5 |
| Film Forming System: | 40.0 |
| Hydroxypropyl methylcellulose (e.g. HPMC of 3 cP, such | |
| as HPMC 2910-3, e.g. 95% w/w) | |
| Polyethylene glycol (e.g. PEG 8000, e.g. 5% w/w) | |
| L-Arginine | 10.0 |
| Polyethylene glycol (e.g. PEG 6000) | 10.0 |
| Talc | 9.0 |
| Purified water | —* |

*Processing agent water used in manufacturing and removed by drying

TABLE 6b
Further Example formulations for extended release core comprising
metformin hydrochloride, barrier coating and API-coating of linagliptin

| Component | mg/tablet |
|---|---|
| Extended release core | |
| Metformin hydrochloride (Metformin HCl) | 1000 |
| Polyethylene Oxide | 370 |
| (e.g. high molecular weight, e.g. about 7,000,000, such as | |
| WSR-303) | |
| Hydroxypropyl methylcellulose (Hypromellose) | 34.0 |
| (e.g. viscosity about 5 mPas for a 2% solution in water at | |
| 20° C., such as HPMC 2910-5 (Methocel ™ E5)) | |
| Magnesium Stearate | 15.0 |
| Barrier coat: | |
| Film Forming System: | 85.0 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 2.5 |
| Film Forming System: | 40.0 |
| Hydroxypropyl methylcellulose (e.g. HPMC of 3 cP, such | |
| as HPMC 2910-3, e.g. 95% w/w) | |
| Polyethylene glycol (e.g. PEG 8000, e.g. 5% w/w) | |
| L-Arginine | 10.0 |
| Polyethylene glycol (e.g. PEG 6000) | 10.0 |
| Talc | 9.0 |
| Purified water | —* |

*Processing agent water used in manufacturing and removed by drying

TABLE 6c
Further Example formulations for extended release core comprising metformin
hydrochloride, barrier coating and API-coating of linagliptin

| Component | mg/tablet |
|---|---|
| Extended release core | |
| Metformin hydrochloride (Metformin HCl) | 1000 |
| Polyethylene Oxide | 370 |
| (e.g. high molecular weight, e.g. about 7,000,000, such as | |
| WSR-303) | |
| Hydroxypropyl methylcellulose (Hypromellose) | 34.0 |
| (e.g. viscosity about 5 mPas for a 2% solution in water at | |
| 20° C., such as HPMC 2910-5 (Methocel ™ E5)) | |
| Magnesium Stearate | 15.0 |
| Barrier coat: | |
| Film Forming System: | 85.0 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 5.0 |
| Film Forming System: | 40.0 |
| Hydroxypropyl methylcellulose (e.g. HPMC of 3 cP, such | |
| as HPMC 2910-3, e.g. 95% w/w) | |
| Polyethylene glycol (e.g. PEG 8000, e.g. 5% w/w) | |
| L-Arginine | 20.0 |
| Polyethylene glycol (e.g. PEG 6000) | 10.0 |
| Talc | 9.0 |
| Purified water | —* |

*Processing agent water used in manufacturing and removed by drying

In the examples according to the Tables 3, 4, 5, 6a, 6b and 6c, as to the barrier coating, the parts titanium dioxide, talc, hydroxypropyl methylcellulose (e.g. 3 cP), hydroxypropyl cellulose, may be employed together (such as e.g. as a film coating system) or separately in the preparation of the barrier coating.

In addition, as to the API coating, the parts hydroxypropyl methylcellulose (e.g. 3 cP) and polyethylene glycol (e.g. PEG 8000) may be employed together (such as e.g. as a film coating system) or separately in the preparation of the API coating.

In the examples according to the Tables 3, 4, 5, 6a, 6b, 6c the barrier coat (solid content) may have a weight in the range from 50 to 100 mg, for example 60-75 mg such as in the examples of Table 6a and 75-95 mg such as in the examples of Table 6b or 6c.

The barrier coat may represent about 1-20% w/w, preferably from about 5% w/w to about 15% w/w (such as e.g. 6% w/w or 12% w/w) of the total composition.

In a particular embodiment, the barrier coat represents about 5-6% w/w of the total composition.

Therefore according to a preferred embodiment of the present invention there is provided a pharmaceutical composition comprising

a) an extended release core comprising metformin (particularly metformin hydrochloride) and one or more excipients;

b) an optional seal and/or barrier coating, preferably barrier coating;

c) an immediate release coating (API coating) comprising polyethylene glycol (e.g. polyethylene glycol of molecular weight of about 6000 and/or 8000,

e.g. 8-25% w/w, preferably 10-20% w/w, such as 16.8 or 14.3% w/w), linagliptin (as API, e.g. 1-10% w/w, preferably 2-7% w/w, such as 3.5 or 6.0% w/w), L-arginine (as stabilizer, e.g. the weight ratio L-arginine:linagliptin may be 1:1 to 5:1, preferably 2:1 to 4:1, especially 4:1; such as e.g. 4-40% w/w, preferably 10-30% w/w, such as 14 or 24% w/w),

hydroxypropyl methylcellulose (e.g. hydroxypropyl methylcellulose of 3 cP, e.g. 40-70% w/w, preferably 40-65% w/w, such as 53.2 or 45.2% w/w),

talc (e.g. 5-20% w/w, preferably 8-16% w/w, such as 12.5 or 10.7% w/w),

wherein % w/w relate to the weight of the immediate release coating.

Preferably the pharmaceutical composition according to this embodiment additionally comprises one or more optional color and/or final coatings.

According to a more preferred embodiment of the present invention there is provided a pharmaceutical composition comprising

a) an extended release core comprising metformin (particularly metformin hydrochloride) and one or more excipients;

b) an optional seal and/or barrier coating, preferably barrier coating;

c) an immediate release coating (API coating) comprising polyethylene glycol of molecular weight of about 6000 (e.g. 8-20% w/w, preferably 10-18 w/w, such as 14.0 or 11.9% w/w),

linagliptin (as API, e.g. 1-10% w/w, preferably 2-7% w/w, such as 3.5 or 6.0% w/w), L-arginine (as stabilizer, e.g. the weight ratio L-arginine: linagliptin may be 1:1 to 5:1,

preferably 2:1 to 4:1, especially 4:1; such as e.g. 4-40% w/w, preferably 10-30% w/w, such as 14 or 24% w/w),

hydroxypropyl methylcellulose (e.g. hydroxypropyl methylcellulose of 3 cP, e.g. 40-70% w/w, preferably 40-65% w/w, such as 53.2 or 45.2% w/w),

polyethylene glycol of molecular weight of about 8000 (e.g. 0-5% w/w, preferably 2-4% w/w, such as 2.8 or 2.4% w/w),

talc (e.g. 5-20% w/w, preferably 8-16% w/w, such as 12.5 or 10.7% w/w), wherein % w/w relate to the weight of the immediate release coating.

Preferably the pharmaceutical composition according to this embodiment additionally comprises one or more optional color and/or final coatings.

According to an even more preferred embodiment of the present invention there is provided a pharmaceutical composition comprising or consisting essentially of

a) an extended release core comprising or consisting essentially of metformin (particularly metformin hydrochloride) and one or more excipients;

b) an optional seal and/or barrier coating, preferably barrier coating;

c) an immediate release coating (API coating) comprising or consisting essentially of polyethylene glycol of molecular weight of about 6000 (e.g. 8-20% w/w, preferably 10-18 w/w, such as 14.0 or 11.9% w/w),

linagliptin (as API, e.g. 1-10% w/w, preferably 2-7% w/w, such as 3.5 or 6.0% w/w),

L-arginine (e.g. the weight ratio L-arginine: linagliptin may be 1:1 to 5:1, preferably 2:1 to 4:1, especially 4:1; such as e.g. 4-40% w/w, preferably 10-30% w/w, such as 14 or 24% w/w), hydroxypropyl methylcellulose (e.g. hydroxypropyl methylcellulose of 3 cP, e.g. 40-70% w/w, preferably 40-65% w/w, such as 53.2 or 45.2% w/w),

polyethylene glycol of molecular weight of about 8000 (e.g. 0-5% w/w, preferably 2-4% w/w, such as 2.8 or 2.4% w/w),

talc (e.g. 5-20% w/w, preferably 8-16% w/w, such as 12.5 or 10.7% w/w),

wherein % w/w relate to the weight of the immediate release coating, and

wherein the content of linagliptin is in the range from 1 to 10 mg, particularly 2.5 mg or 5.0 mg.

Preferably the pharmaceutical composition according to this embodiment additionally comprises one or more optional color and/or final coatings.

According to a further more preferred embodiment of the present invention there is provided a pharmaceutical composition comprising

a) an extended release core comprising

metformin (particularly metformin hydrochloride)

hydroxypropyl methylcellulose (e.g. viscosity about 5 mPas for a 2% solution in water at 20° C., such as Methocel™ E5, e.g. 2-3% w/w),

polyethylene oxide (e.g. high molecular weight, e.g. about 7,000,000, such as WSR-303, e.g. 23-35% w/w),

magnesium stearate (e.g. 0.5-2% w/w),

wherein % w/w relate to the weight of the extended release core, and

wherein the content of metformin hydrochloride is in the range from 500 to 1000 mg, for example 500, 750 or 1000 mg;

b) a barrier coating, e.g. comprising hydroxypropyl cellulose, hydroxypropyl methylcellulose (e.g. 3 cP, such as HPMC 2910 3 cP), titanium dioxide and talc;

c) an immediate release coating comprising

polyethylene glycol of molecular weight of about 6000 (e.g. 8-20% w/w, preferably 10-18 w/w, such as 14.0 or 11.9% w/w),

linagliptin (e.g. 1-10% w/w, preferably 2-7% w/w, such as 3.5 or 6.0% w/w), L-arginine (e.g. the weight ratio L-arginine:linagliptin may be 1:1 to 5:1, preferably 2:1 to 4:1, especially 4:1; such as e.g. 4-40% w/w, preferably 10-30% w/w, such as 14 or 24% w/w), hydroxypropyl methylcellulose (e.g. hydroxypropyl methylcellulose of 3 cP, e.g. 40-70% w/w,

preferably 40-65% w/w, such as 53.2 or 45.2% w/w),

polyethylene glycol of molecular weight of about 8000 (e.g. 0-5% w/w, preferably 2-4% w/w, such as 2.8 or 2.4% w/w),

talc (e.g. 5-20% w/w, preferably 8-16% w/w, such as 12.5 or 10.7% w/w),

wherein % w/w relate to the weight of the immediate release coating, and

wherein the content of linagliptin is in the range from 1 to 10 mg, particularly 2.5 mg or 5.0 mg.

Preferably the pharmaceutical composition according to this embodiment additionally comprises one or more optional color and/or final coatings.

Film coating suspensions/solutions of API (linagliptin) according to this invention can be prepared by common methods, such as follows:

The film-coating agent hydroxypropyl methylcellulose (HPMC), the plasticizer polyethylene glycol (PEG) (e.g. Macrogol 400, 6000 or 8000) or, as alternative plasticizer, propylene glycol (PG) and water are dissolved and mixed by a suitable mixer (e.g. by propeller mixer) to produce the API-free coating solution. Optionally, the glidant talc suspended in water is added and the obtained suspension is homogenized. Talc may be used optionally.

The API (linagliptin) and—preferably in case of linagliptin—the stabilizer L-arginine are dissolved or suspended in water and added to the aqueous solution of HPMC, PEG or PG, and, optional talc, and dispersed by a suitable mixer (e.g. by propeller mixer) to provide the API coating suspension.

Alternatively, the film-coating agent hydroxypropyl methylcellulose (HPMC) and water are dissolved and mixed by a suitable mixer (e.g. by Ultraturrax®).

The stabilizer L-arginine (which is present in case of linagliptin), the plasticizer polyethylene glycol (PEG) (e.g. Macrogol 400, 6000 or 8000) or propylene glycol (PG), optional talc, and water are dispersed, e.g. by homogenization using e.g. Ultraturrax®.

After degassing of the HPMC solution (or directly after manufacturing of the HPMC solution), the aqueous suspension of PEG or PG, optional L-arginine and optional talc are added to the aqueous HPMC solution and mixed/homogenized.

The API (linagliptin) is dissolved or suspended in water and added to the aqueous solution of HPMC, PEG or PG, optional L-arginine and optional talc to provide the API coating suspension.

The film-coating operation is carried out in a conventional film coater. The film coating suspension and/or API (linagliptin) coating suspension/solution are coated at (optionally seal and/or barrier coated) metformin XR cores via coating process.

Preliminary preheating of the cores may be necessary, due to need of equilibrium of water amount of the cores.

The spray rate and air flow through the coating pan is adjusted to produce a uniform coating and coverage of the entire width of the tablet bed. The amount of the coating suspension applied can be controlled by percent weight gain of tablet cores and typically ranges from about 4 to about 12.5%.

In one aspect, this range results in linagliptin drug assay close to the desired 2.5 mg or 5 mg with a standard deviation of about 2-4% for content uniformity assay of linagliptin. The duration of the coating step is about 4-10 hours. The duration of the coating step depends on batch size, process parameters like spray rate and solid concentrations of the coating suspension.

The API coating suspension is applied to the (optionally seal and/or barrier coated) tablet cores containing the metformin XR formulation and the amount of solids deposited in the API film layer is controlled to achieve the desired API doses.

The weight of the cores and film coated tablets may be controlled by percent weight gain during the coating process. Instead of or in addition to weight gain method a PAT method, e.g. online NIR or Raman method for end point detection of assay of API may be used.

### Further Embodiments

An optional seal and/or barrier coat (preferably a barrier coat) may separate the metformin XR core from the API-containing film coat. Typically, for the preparation of film-coated tablets a coating suspension is prepared and the tablet cores may be coated with the seal coating suspension and/or with the barrier coating suspension using standard film coater.

The film coating solvent is a volatile component, which does not remain in the final product. A typical seal and/or barrier film-coat comprises a film coating agent, a plasticizer, and, optionally, a glidant, one or more pigments and/or colors.

The metformin XR core may be seal and/or barrier coated using a seal coating agent (and a plasticizer) and/or a barrier coating agent (and a plasticizer), such as with a mixture of hydroxypropylcellulose and hydroxypropyl methylcellulose, a mixture of polyvinyl alcohol (PVA) and polyethylene glycol (PEG), a mixture of hydroxypropyl methylcellulose and either polyethylene glycol (PEG) or propylene glycol (PG), or any other suitable immediate-release film-coating agent(s). A commercial film-coat is Opadry®, Opadry II® or other Opardy® IR film coat, which are formulated powder blend provided by Colorcon®. Optionally the seal coat may further comprise a glidant. Optionally the barrier coat may further comprise a glidant.

Preferably, the barrier coat of this invention comprises or consists essentially of hydroxypropyl cellulose and hydroxypropyl methylcellulose.

For example, a seal or barrier coat may comprise or consist essentially of a film-coating agent which is hydroxypropyl methylcellulose (HPMC, e.g. hypromellose 2910 such as with nominal viscosity of 3 cP), with or without hydroxypropyl cellulose (HPC, e.g. HPC-EF),

optionally, a plasticizer which may be polyethylene glycol,

optionally, a glidant which is talc, and

optionally one or more pigments and/or colors.

For further example, a seal or barrier coat may comprise or consist essentially of one or more film-coating agents selected from hydroxypropyl methylcellulose (HPMC, hypromellose such as e.g. hypromellose 2910 with nominal viscosity of 3 cP, hypromellose 2910 with nominal viscosity of 5 cP, hypromellose 2910 with nominal viscosity of 6 cP, hypromellose 2910 with nominal viscosity of 15 cP, hypromellose 2910 with nominal viscosity of 50 cP, Methocel™ E5, Methocel™ E15, Pharmacoat® 603, Pharmacoat® 606, and/or Pharmacoat® 615), hydroxypropyl cellulose, or mixtures thereof,

optionally a plasticizer selected from polyethylene glycol (such as e.g. Macrogol 400, 3000, 4000, 6000 or 8000), and

optionally one or more pigments (e.g. titanium dioxide), glidants (e.g. talc) and/or colors.

For further example, a seal or barrier coat may comprise or consist essentially of one or more hydroxypropyl methylcellulose (HPMC, hypromellose 2910 with nominal viscosity of 3 cP, hypromellose 2910 with nominal viscosity of 6 cP, hypromellose 2910 with nominal viscosity of 50 cP) with or without hydroxypropyl cellulose,

optionally a plasticizer (which may be polyethylene glycol 8000), and

optionally one or more pigments (e.g. titanium dioxide), glidants (e.g. talc) and/or colors.

For further example, a seal or barrier coat may comprise or consist essentially of a mixture of one or more hydroxypropyl methylcellulose (HPMC, hypromellose 2910 with nominal viscosity of 3 cP, hypromellose 2910 with nominal viscosity of 6 cP, hypromellose 2910 with nominal viscosity of 50 cP) and hydroxypropyl cellulose, optionally one or more pigments (e.g. titanium dioxide), glidants (e.g. talc) and/or colors.

For further example, a barrier coating may comprise or consist essentially of hydroxypropyl cellulose (e.g. HPC-EF), hydroxypropyl methylcelluloses (e.g. 3 to 50 cP, such as HPMC 2910-3, HPMC 2910-6 and/or HPMC 2910-50), titanium dioxide, and talc, and wherein the barrier coating may have a weight for example in the range from 50 to 100 mg (such e.g. 68 mg or 85 mg).

For example, a seal or barrier coat comprises or consists essentially of

a film-coating agent which is hydroxypropyl methylcellulose (HPMC, hypromellose such as e.g. HPMC 2910 3 cP), optionally together with hydroxypropyl cellulose (HPC, e.g. HPC-EF),

optionally, a glidant which is talc, and

optionally, a pigment (e.g. titanium dioxide).

Preferably, in a particular embodiment, a barrier coating according to this invention comprises or consists essentially of

hydroxypropyl methylcellulose (e.g. of 3 cP, such as HPMC 2910 3 cP), hydroxypropyl cellulose, titanium dioxide and talc,

wherein the barrier coating may have a weight in the range from 50 to 100 mg (such e.g. 68 mg or 85 mg), and/or

wherein the barrier coating may represent from about 5% w/w to about 15% w/w (such as e.g. 6% w/w or 12% w/w) of the total composition.

In a further embodiment, the ratio of hydroxypropyl methylcellulose: hydroxypropyl cellulose may be from about 10:1 to about 1:10, such as e.g. about 1:1.

For example, the barrier coating suspension is an aqueous suspension (e.g. 15% w/w) of Film Forming System (Table A, B or C) comprising or consisting essentially of hydroxypropyl cellulose (e.g. 33.75% w/w), hydroxypropyl methylcellulose (e.g. HPMC of 3 cP, such as Hypromellose 2910 3 cP, e.g. 33.75% w/w), talc (e.g. 20.0% w/w) and titanium dioxide (e.g. 12.5% w/w), which can be used to form the barrier coat such as to separate the metformin XR tablet core from the API containing film coat.

### Further Embodiments

The final pharmaceutical compositions of the present invention are tablets. Such tablets may be further film-coated with a color and/or final film over-coat, such as with a mixture of hydroxypropylcellulose and hydroxypropyl methylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; a mixture of hydroxypropyl methylcellulose and either polyethylene glycol (PEG) or propylene glycol (PG) containing titanium dioxide and/or other coloring agents, such as iron oxides, dyes, and lakes; or any other suitable immediate-release film-coating agent(s). Optionally a glidant is present. The coat may provide taste masking and additional stability to the final tablet. A commercial film-coat is Opadry®, Opadry II® or other Opardy® IR film coat, which are formulated powder blend provided by Colorcon®.

### Yet Further Embodiments

Preferably, for the preparation of film-coated tablets a coating suspension is prepared and the tablet cores are coated with the coating suspension, typically for the API-free film coat to a weight gain of about 2-4%, preferably about 3%, using standard film coater. The film coating solvent is a volatile component, which does not remain in the final product. A typical film-coat comprise a film coating agent, a plasticizer, and, optionally, a glidant, one or more pigments and/or colors.

For example, the film coat may comprise hydroxypropylmethylcellulose (HPMC), propylene glycol or polyethylene glycol, talc and, optionally, titanium dioxide and/or iron oxide (e.g. iron oxide yellow and/or red).

For further example, the film coat may comprise hydroxypropylmethylcellulose (HPMC), hydroxypropylcellulose (HPC), talc and, optionally, titanium dioxide.

In further embodiments of the film coating layers of this invention, the film-coating agent may be one or more of hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), polyvinyl alcohol (PVA), ethyl cellulose, hydroxypropyl cellulose (HPC), polydextrose, methacrylic and/or acrylic polymer, or mixtures thereof (e.g. a mixture of one or more HPMC and polydextrose, or a mixture of a HPMC and HPC).

In an embodiment of the film coating layers of this invention, the film-coating agent may be one or more hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615).

In a particular embodiment of the film coating layers of this invention, the film-coating agent may be a hydroxypropyl methylcellulose (HPMC, hypromellose such as e.g. hypromellose 2910 with nominal viscosity of 3 or 6 cP).

In another particular embodiment of the film coating layers of this invention, the film-coating agent may be a mixture of hydroxypropyl methylcellulose (HPMC, hypromellose such as e.g. hypromellose 2910 with nominal viscosity of 3 cP) and hydroxypropyl cellulose.

In further embodiments of the film coating layers of this invention, the plasticizer may be one or more of polyethylene glycol (such as e.g. Macrogol 400, 3000, 4000, 6000 and/or 8000), propylene glycol, diethyl phthalate, tributyl sebacate and/or triacetin, or mixtures thereof (e.g. a mixture of polyethylene glycol and triacetin).

In a particular embodiment of the film coating layers of this invention, the plasticizer may be a polyethylene glycol (such as e.g. PEG 8000).

The plasticizer may be optional.

In further embodiments of the film coating layers of this invention, the glidant may be one or more of talc, magnesium stearate and/or fumed silica, or mixtures thereof.

In a particular embodiment of the film coating layers of this invention, the glidant may be talc.

The glidant may be optional.

In a particular embodiment of the film coating layers of this invention, the pigment may be titanium dioxide.

The pigment and/or color may be optional.

In certain embodiments of the film coating layers of this invention, the film-coating agent may be hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), and the plasticizer may be polyethylene glycol (such as e.g. Macrogol 400, 3000, 4000, 6000 or 8000).

In certain embodiments of the film coating layers of this invention, the film-coating agent may be hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), and hydroxypropyl cellulose.

In certain other embodiments of the film coating layers, the film-coating agent may be hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), and the plasticizer may be triacetin.

In certain other embodiments of the film coating layers, the film-coating agent may be a mixture of one or more hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615) and polydextrose, and the plasticizer may be polyethylene glycol (such as e.g. Macrogol 400, 3000, 4000, 6000 or 8000).

In certain other embodiments of the film coating layers, the film-coating agent may be polyvinyl alcohol (PVA), and the plasticizer may be polyethylene glycol (such as e.g. Macrogol 400, 3000, 4000, 6000 or 8000).

In certain other embodiments of the film coating layers, the film-coating agent may be polyvinyl alcohol (PVA), and the plasticizer may be triacetin.

In a further embodiment of this invention, a film coat according to this invention may comprise or consist essentially of

one or more film-coating agents, e.g. selected from hydroxypropyl methylcellulose (HPMC, hypromellose such as e.g. hypromellose 2910 with nominal viscosity of 3, 5, 6, 15 and/or 50 cP, Methocel™ E5, Methocel™ E15, Pharmacoat® 603, Pharmacoat® 606, and/or Pharmacoat® 615), polyvinyl alcohol (PVA), ethyl cellulose, hydroxypropyl cellulose, polydextrose, methacrylic and/or acrylic polymer, or a mixture thereof,

optionally one or more plasticizers, e.g. selected from polyethylene glycol (such as e.g. Macrogol 400, 3000, 4000, 6000 and/or 8000), propylene glycol, diethyl phthalate, tributyl sebacate and triacetin, or a mixture thereof,

optionally a glidant, e.g. talc, magnesium stearate or fumed silica, and

optionally one or more pigments (e.g titanium dioxide) and/or colors (e.g. based on iron oxides).

Accordingly, the film coating layers according to this invention may comprise one or more of the following:

at least one film-coating agent, such as e.g. hydroxypropyl methylcellulose (HPMC, hypromellose such as e.g. hypromellose 2910 with nominal viscosity of 3, 5, 6, 15 or 50 cP, Methocel™ E5, Methocel™ E15, Pharmacoat® 603, Pharmacoat® 606, or Pharmacoat® 615), polyvinyl alcohol (PVA), ethyl cellulose, hydroxypropyl cellulose, polydextrose, or a methacrylic or acrylic polymer,

optionally, at least one plasticizer, such as e.g. polyethylene glycol (such as e.g. Macrogol 400, 3000, 4000, 6000 or 8000), propylene glycol, diethyl phthalate, tributyl sebacate or triacetin,

at least one glidant or anti-adherent, such as e.g. talc, magnesium stearate or fumed silica, and

at least one pigment, such as e.g. titanium dioxide, and/or colorant (such as e.g. based on an iron oxide).

In certain embodiments, the hydroxypropyl methylcellulose (HPMC) as film-coating agent is hypromellose having viscosity from about 3 centipoise to about 15 centipoise or up to 40-60 centipoise, measured as aqueous 2% solution at 20° C., e.g. 3, 5, 6, 15 or 50 cP, such as e.g. hypromellose 2910 with nominal viscosity of 3 cP, hypromellose 2910 with nominal viscosity of 5 cP, hypromellose 2910 with nominal viscosity of 6 cP, hypromellose 2910 with nominal viscosity of 15 cP, hypromellose 2910 with nominal viscosity of 50 cP, Methocel™ E5, Methocel™ E15, Pharmacoat® 603, Pharmacoat® 606, or Pharmacoat® 615.

In certain embodiments, the polyethylene glycol (PEG) as plasticizer is macrogol having average molecular weight from about 400 to about 8000 daltons, e.g. 400, 1500, 3000, 4000, 6000 or 8000 D, such as e.g. Marcrogol 400, Marcrogol 6000 or Marcrogol 8000.

In certain embodiments, the coating material is commercially available under the trade name Opadry®, Opadry II® or other Opadry® film coat.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes.

The pharmaceutical compositions (or formulations) may be packaged in a variety of ways. Generally, an article for distribution includes a container that contains the pharmaceutical composition in an appropriate form. Tablets are typically packed in an appropriate primary package for easy handling, distribution and storage and for assurance of proper stability of the composition at prolonged contact with the environment during storage. Primary containers for tablets may be bottles or blister packs, optionally with desiccant.

A suitable bottle may be made from glass or polymer (preferably polypropylene (PP) or high density polyethylene (HD-PE)) and sealed with a screw cap. The screw cap may be provided with a child resistant safety closure (e.g. press-and-twist closure) for preventing or hampering access to the contents by children. If required (e.g. in regions with high humidity), by the additional use of a desiccant (such as e.g. bentonite clay, molecular sieves, or, preferably, silica gel) the shelf life of the packaged composition can be prolonged.

A suitable blister pack comprises or is formed of a top foil (which is breachable by the tablets) and a bottom part (which contains pockets for the tablets). The top foil may contain a metalic foil, particularly an aluminium or aluminium alloy foil (e.g. having a thickness of 20 μm to 45 μm, preferably 20 μm to 25 μm) that is coated with a heat-sealing polymer layer on its inner side (sealing side). The bottom part may contain a multi-layer polymer foil (such as e.g. poly(vinyl choride) (PVC) coated with poly(vinylidene choride) (PVDC); or a PVC foil laminated with poly(chlorotriflouroethylene) (PCTFE)) or a multi-layer polymer-metal-polymer foil (such as e.g. a cold-formable laminated PVC/aluminium/polyamide composition).

To ensure a long storage period especially under hot and wet climate conditions an additional overwrap or pouch made of a multi-layer polymer-metal-polymer foil (e.g. a laminated polyethylen/aluminium/polyester composition) may be used for the blister packs. Supplementary desiccant (such as e.g. bentonite clay, molecular sieves, or, preferably, silica gel) such as in this pouch package may prolong the shelf life even more under such harsh conditions.

The article may further comprise a label or package insert, which refer to instructions customarily included in commercial packages of therapeutic products, that may contain information about the indications, usage, dosage, administration, contraindications and/or warnings concerning the use of such therapeutic products. In one embodiment, the label or package inserts indicates that the composition can be used for any of the purposes described herein.

The present invention also provides methods particularly for treating type 2 diabetes by orally administering to a host in need of such treatment a therapeutically effective amount of one of the fixed-dose combination pharmaceutical compositions of the present invention. In one embodiment the host in need of such treatment is a human. In another embodiment the pharmaceutical composition is in the dosage form of a tablet. The pharmaceutical compositions comprising the fixed-dose combination may be administered once-daily (QD), twice-daily (BID), thrice-daily (TID), or four-times daily.

Further, illustrative effects of the compositions according to the present invention are provided as follows, for example:

Five batches of linagliptin/metformin (e.g. 2.5/1000 mg) and different ratios (w/w) of linagliptin:arginine (1:1 to 1:5) are stored open/closed at 40° C./75% r.h. over 3 months. Overall, under closed storage conditions, no significant change in linagliptin assay results in all arginine-containing batches observed. Likewise, under closed storage conditions, no significant changes in linagliptin and metformin dissolution results are observed in the arginine-containing batches. Under open storage conditions, a correlation between arginine content and linagliptin assay results can be observed; based thereon, a linagliptin/arginine ratio of 1:4 (w/w) is selected as optimum.

In an embodiment, pharmaceutical dosage forms of this invention (preferably containing metformin XR and linagliptin as API) preferably have dissolution properties such that, for example, after 2 hours 31-54% by weight of the metformin HCl active ingredient is dissolved, and/or after 4 hours 51-74% by weight of the metformin HCl active ingredient is dissolved, and/or after 12 hours not less than 80% by weight of the metformin HCl active ingredient is dissolved.

In a further embodiment, pharmaceutical dosage forms of this invention (preferably containing metformin XR and linagliptin as API) preferably have dissolution properties such that after 45 minutes at least 75%, or at least 80%, or at least 90% by weight of linagliptin is dissolved. In a particular embodiment, after 30 minutes for linagliptin at least 70-80 (preferably not less than 75%) by weight of linagliptin is dissolved.

The dissolution properties can be determined in standard dissolution tests, e.g. according to standard pharmacopeias (such as e.g. using paddle/basket method with agitation speed of 50 rpm or 100 rpm, pH1.0 (HCl/SGF), and HPLC (linagliptin) and UV (metformin) analysis of the samples).

For illustrative example, in a certain embodiment, at pH 1 (HCl/SGF) the dissolution profiles of a linagliptin+metformin XR FDC of this invention (such as e.g. FDC 2.5/750 mg, FDC 5/1000 mg, FDC 2.5/1000 mg) and the respective reference innovator products (Trajenta® and Glumetza®) are preferably similar (f2 ≧50) or in the same range, using e.g. the innovator dissolution method (metformin: 100 rpm basket, linagliptin: 50 rpm basket; pH 1).

In a certain embodiment, the linagliptin+metformin XR FDC of this invention (5 mg/1000 mg, 1×) is bioequivalent (for linagliptin) to linagliptin 5 mg tablet (Trajenta®), and (for metformin) to 2×Glumetza® 500 mg.

In a certain embodiment, the linagliptin+metformin XR FDC of this invention (2.5 mg/1000 mg, 2×) is bioequivalent (for linagliptin) to linagliptin 5 mg tablet (Trajenta®), and (for metformin) to 4× Glumetza® 500 mg.

In a certain embodiment, the linagliptin+metformin XR FDC of this invention (2.5 mg/750 mg, 2×) is bioequivalent (for linagliptin) to linagliptin 5 mg tablet (Trajenta®), and (for metformin) to 3× Glumetza® 500 mg.

The barrier coat according to the present invention comprising or consisting essentially of hydroxypropyl cellulose, hydroxypropyl methylcellulose (e.g. HPMC 3 cP), titanium dioxide and talc (Barrier coat A) is optimized for stability and provides improved protection for arginine and linagliptin, such as compared to the barrier coat comprising or consisting essentially of hydroxypropyl methylcellulose (e.g. HPMC 3 cP, HPMC 6 cP, HPMC 50 cP), polyethylene glycol (PEG 8000), titanium dioxide and polydextrose (Barrier coat B).

For example, illustrative results for stability using the different Barrier coats A and B (each 6%/12% coating levels) evaluated under stress conditions (40° C., 75% rh, closed/open container, 6 months storage) can be found in the following table:

| | Barrier coat A | Barrier coat B |
|---|---|---|
| Linagliptin content at start | about 100% | about 100% |
| Linagliptin content after 6 | ~subst. unchanged | about 55% |
| months storage, 40° C., 75% rh, | (6% coating level) | (6% coating level) |
| open container | ~subst. unchanged | about 40% |
| | (12% coating level) | (12% coating level) |

Barrier coat A further provides very good coating performance.

Manufacture and Polymorph

The term “linagliptin” as employed herein refers to linagliptin, a pharmaceutically acceptable salt thereof, a hydrate or solvate thereof, or a polymorphic form thereof. Crystalline forms are described in WO 2007/128721. Preferred crystalline forms are the polymorphs A and B described therein. In particular, linagliptin is the free base 1-[(4-methyl-quinazolin-2-yl)methyl]-3-methyl-7-(2-butyn-1-yl)-8-(3-(R)-amino-piperidin-1-yl)-xanthine. As linagliptin or a pharmaceutically acceptable salt thereof, linagliptin is preferred. Methods for the manufacture of linagliptin are described in the patent applications WO 2004/018468 and WO 2006/048427 for example. A therapeutic dose of linagliptin may be 5 mg per patient per day. 1-[(4-Methyl-quinazolin-2-yl)methyl]-3-methyl-7-(2-butyn-1-yl)-8-(3-(R)-amino-piperidin-1-yl)-xanthine (linagliptin):

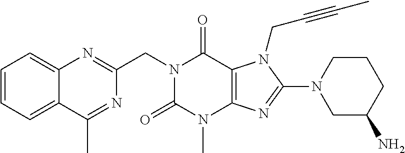 [SMILES: CC#CCn1c(N2CCC[C@@H](N)C2)nc2c1c(=O)n(Cc1nc(C)c3ccccc3n1)c(=O)n2C]

For avoidance of any doubt, the disclosure of each of the foregoing documents cited above in connection with the specified DPP-4 inhibitors is specifically incorporated herein by reference in its entirety.

Indications

As described herein by the administration of the pharmaceutical composition according to this invention, therapeutic effects can be achieved, which make it useful for treating and/or preventing certain diseases, disorders or conditions, such as e.g. those described herein.

Therefore, a treatment or prophylaxis according to this invention is advantageously suitable in those patients in need of such treatment or prophylaxis who are diagnosed of one or more of the conditions selected from the group consisting of overweight and obesity, in particular class I obesity, class II obesity, class III obesity, visceral obesity and abdominal obesity. In addition a treatment or prophylaxis according to this invention is advantageously suitable in those patients in which a weight increase is contraindicated. The pharmaceutical composition as well as the methods according to the present invention allow a reduction of the HbA1c value to a desired target range, for example <7% and preferably <6.5%, for a higher number of patients and for a longer time of therapeutic treatment compared with a corresponding monotherapy.

The pharmaceutical composition according to this invention and in particular the active ingredients therein exhibits a very good efficacy with regard to glycemic control, in particular in view of a reduction of fasting plasma glucose, postprandial plasma glucose and/or glycosylated hemoglobin (HbA1c). By administering a pharmaceutical composition according to this invention, a reduction of HbA1c equal to or greater than preferably 0.5%, even more preferably equal to or greater than 1.0% can be achieved and the reduction is particularly in the range from 1.0% to 2.0%.

Furthermore, the method and/or use according to this invention is advantageously applicable in those patients who show one, two or more of the following conditions:
(a) a fasting blood glucose or serum glucose concentration greater than 110 mg/dL, in particular greater than 125 mg/dL;
(b) a postprandial plasma glucose equal to or greater than 140 mg/dL;
(c) an HbA1c value equal to or greater than 6.5%, in particular equal to or greater than 7.0%, especially equal to or greater than 7.5%, even more particularly equal to or greater than 8.0%.

The present invention also discloses the use of the pharmaceutical composition for improving glycemic control in patients having type 2 diabetes or showing first signs of pre-diabetes. Thus, the invention also includes diabetes prevention. If therefore a pharmaceutical composition according to this invention is used to improve the glycemic control as soon as one of the above-mentioned signs of pre-diabetes is present, the onset of manifest type 2 diabetes mellitus can be delayed or prevented.

Furthermore, the pharmaceutical composition according to this invention is particularly suitable in the treatment of patients with insulin dependency, i.e. in patients who are treated or otherwise would be treated or need treatment with an insulin or a derivative of insulin or a substitute of insulin or a formulation comprising an insulin or a derivative or substitute thereof. These patients include patients with diabetes type 2 and patients with diabetes type 1.

Therefore, according to a preferred embodiment of the present invention, there is provided a method for improving glycemic control and/or for reducing of fasting plasma glucose, of postprandial plasma glucose and/or of glycosylated hemoglobin HbA1c in a patient in need thereof who is diagnosed with impaired glucose tolerance (IGT), impaired fasting blood glucose (IFG) with insulin resistance, with metabolic syndrome and/or with type 2 or type 1 diabetes mellitus characterized in that a pharmaceutical composition as defined hereinbefore and hereinafter is administered to the patient.

According to another preferred embodiment of the present invention, there is provided a method for improving gycemic control in patients, in particular in adult patients, with type 2 diabetes mellitus as an adjunct to diet and exercise.

Therefore, the method and/or use according to this invention is advantageously applicable in those patients who show one, two or more of the following conditions:
(a) insufficient glycemic control with diet and exercise alone;
(b) insufficient glycemic control despite oral monotherapy with metformin, in particular despite oral monotherapy at a maximal tolerated dose of metformin;
(c) insufficient glycemic control despite oral monotherapy with another antidiabetic agent, in particular despite oral monotherapy at a maximal tolerated dose of the other antidiabetic agent.

The lowering of the blood glucose level by the administration of a pharmaceutical composition according to this invention is insulin-independent. Therefore, a pharmaceutical composition according to this invention is particularly suitable in the treatment of patients who are diagnosed having one or more of the following conditions
insulin resistance,
hyperinsulinemia,
pre-diabetes,
type 2 diabetes mellitus, particular having a late stage type 2 diabetes mellitus,
type 1 diabetes mellitus.

Furthermore, a pharmaceutical composition according to this invention is particularly suitable in the treatment of patients who are diagnosed having one or more of the following conditions
(a) obesity (including class I, II and/or III obesity), visceral obesity and/or abdominal obesity,
(b) triglyceride blood level ≧150 mg/dL,
(c) HDL-cholesterol blood level <40 mg/dL in female patients and <50 mg/dL in male patients,
(d) a systolic blood pressure ≧130 mm Hg and a diastolic blood pressure ≧85 mm Hg,
(e) a fasting blood glucose level ≧110 mg/dL.

It is assumed that patients diagnosed with impaired glucose tolerance (IGT), impaired fasting blood glucose (IFG), with insulin resistance and/or with metabolic syndrome suffer from an increased risk of developing a cardiovascular disease, such as for example myocardial infarction, coronary heart disease, heart insufficiency, thromboembolic events. A glycemic control according to this invention may result in a reduction of the cardiovascular risks.

A pharmaceutical composition according to this invention exhibits a good safety profile. Therefore, a treatment or prophylaxis according to this invention is advantageously possible in those patients for which the mono-therapy with another antidiabetic drug is contraindicated and/or who have an intolerance against such drugs at therapeutic doses. In particular, a treatment or prophylaxis according to this invention may be advantageously possible in those patients showing or having an increased risk for one or more of the following disorders: renal insufficiency or diseases, cardiac diseases, cardiac failure, hepatic diseases, pulmonal diseases, catabolytic states and/or danger of lactate acidosis, or female patients being pregnant or during lactation.

Furthermore, it can be found that the administration of a pharmaceutical composition according to this invention results in no risk or in a low risk of hypoglycemia. Therefore, a treatment or prophylaxis according to this invention is also advantageously possible in those patients showing or having an increased risk for hypoglycemia.

A pharmaceutical composition according to this invention is particularly suitable in the long term treatment or prophylaxis of the diseases and/or conditions as described hereinbefore and hereinafter, in particular in the long term glycemic control in patients with type 2 diabetes mellitus.

The term “long term” as used hereinbefore and hereinafter indicates a treatment of or administration in a patient within a period of time longer than 12 weeks, preferably longer than 25 weeks, even more preferably longer than 1 year.

Therefore, a particularly preferred embodiment of the present invention provides a method for therapy, preferably oral therapy, for improvement, especially long term improvement, of glycemic control in patients with type 2 diabetes mellitus, especially in patients with late stage type 2 diabetes mellitus, such as e.g. in patients additionally diagnosed of overweight, obesity (including class I, class II and/or class III obesity), visceral obesity and/or abdominal obesity.

According to another aspect of the invention, there is provided a method for preventing, slowing the progression of, delaying or treating of a condition or disorder selected from the group consisting of complications of diabetes mellitus such as cataracts and micro- and macrovascular diseases, such as dyslipidemia, nephropathy, retinopathy, neuropathy, tissue ischaemia, diabetic foot, arteriosclerosis, myocardial infarction, accute coronary syndrome, unstable angina pectoris, stable angina pectoris, stroke, peripheral arterial occlusive disease, cardiomyopathy, heart failure, heart rhythm disorders and vascular restenosis, in a patient in need thereof characterized in that a pharmaceutical composition according to the invention is administered to the patient. In particular one or more aspects of diabetic nephropathy such as hyperperfusion, proteinuria and albuminuria may be treated, their progression slowed or their onset delayed or prevented. The term “tissue ischaemia” particularly comprises diabetic macroangiopathy, diabetic microangiopathy, impaired wound healing and diabetic ulcer. The terms “micro- and macrovascular diseases” and “micro- and macrovascular complications” are used interchangeably in this application.

According to another aspect of the invention, there is provided a method for preventing, slowing the progression of, delaying or treating a metabolic disorder selected from the group consisting of type 2 diabetes mellitus, impaired glucose tolerance (IGT), impaired fasting blood glucose (IFG), hyperglycemia, postprandial hyperglycemia, overweight, obesity, metabolic syndrome, gestational diabetes and diabetes related to cystic fibrosis in a patient in need thereof characterized in that a pharmaceutical composition according to the invention is administered to the patient.

According to another aspect of the invention, there is provided a method for improving glycemic control and/or for reducing of fasting plasma glucose, of postprandial plasma glucose and/or of glycosylated hemoglobin HbA1c in a patient in need thereof characterized in that a a pharmaceutical composition according to the invention is administered to the patient.

The pharmaceutical composition according to this invention may also have valuable disease-modifying properties with respect to diseases or conditions related to impaired glucose tolerance (IGT), impaired fasting blood glucose (IFG), insulin resistance and/or metabolic syndrome.

According to another aspect of the invention, there is provided a method for preventing, slowing, delaying or reversing progression from impaired glucose tolerance (IGT), impaired fasting blood glucose (IFG), insulin resistance and/or from metabolic syndrome to type 2 diabetes mellitus in a patient in need thereof characterized in that a pharmaceutical composition according to the invention is administered to the patient.

As by the use of a pharmaceutical composition according to this invention, an improvement of the glycemic control in patients in need thereof is obtainable, also those conditions and/or diseases related to or caused by an increased blood glucose level may be treated.

By the administration of a pharmaceutical composition according to this invention excessive blood glucose levels are not converted to insoluble storage forms, like fat, but excreted through the urine of the patient. It can be seen that loss of fat may account for the majority of the observed weight loss whereas no significant changes in body water or protein content are observed. Therefore, no gain in weight or even a reduction in body weight is the result.

According to another aspect of the invention, there is provided a method for reducing body weight and/or body fat or preventing an increase in body weight and/or body fat or facilitating a reduction in body weight and/or body fat in a patient in need thereof characterized in that a pharmaceutical composition according to the invention is administered to the patient.

By the administration of a combination or pharmaceutical composition according to the present invention, an abnormal accumulation of ectopic fat, in particular of the liver, may be reduced or inhibited. Therefore, according to another aspect of the present invention, there is provided a method for preventing, slowing, delaying or treating diseases or conditions attributed to an abnormal accumulation of ectopic fat, in particular of the liver, in a patient in need thereof characterized in that a pharmaceutical composition according to the invention is administered to the patient. Diseases or conditions which are attributed to an abnormal accumulation of liver fat are particularly selected from the group consisting of general fatty liver, non-alcoholic fatty liver (NAFL), non-alcoholic steatohepatitis (NASH), hyperalimen-tation-induced fatty liver, diabetic fatty liver, alcoholic-induced fatty liver or toxic fatty liver.

Another aspect of the invention provides a method for maintaining and/or improving the insulin sensitivity and/or for treating or preventing hyperinsulinemia and/or insulin resistance in a patient in need thereof characterized in that a pharmaceutical composition according to the invention is administered to the patient.

According to another aspect of the invention, there is provided a method for preventing, slowing progression of, delaying, or treating new onset diabetes after transplantation (NODAT) and/or post-transplant metabolic syndrome (PTMS) in a patient in need thereof characterized in that a pharmaceutical composition according to the invention is administered to the patient.

According to a further aspect of the invention, there is provided a method for preventing, delaying, or reducing NODAT and/or PTMS associated complications including micro- and macrovascular diseases and events, graft rejection, infection, and death in a patient in need thereof characterized in that a pharmaceutical composition according to the invention is administered to the patient.

Accordingly, the pharmaceutical composition according to present invention can be used in treating and/or preventing (including slowing the progression and/or delaying the onset and/or reducing the risk) of metabolic diseases, especially type 2 diabetes mellitus and conditions related thereto (e.g. diabetic complications, such as micro- and/or macrovascular (cardio- and/or cerebrovascular) complications),

either in type 2 diabetes patients who have not been previously treated with an antihyperglycemic agent (e.g. drug-naïve patients, such as e.g. as first line or initial combination therapy),

or in type 2 diabetes patients with insufficient glycemic control despite therapy with one or two conventional antihyperglycemic agents selected from metformin, sulphonylureas, thiazolidinediones (e.g. pioglitazone), glinides, alpha-glucosidase blockers, GLP-1 or GLP-1 analogues, and insulin or insulin analogues (e.g. previously-treated patients, such as e.g. as second or third line or add-on combination therapy),

optionally in combination with an conventional antihyperglycemic agent.

In an embodiment, the pharmaceutical composition according to present invention can be used in treating and/or preventing (including slowing the progression and/or delaying the onset and/or reducing the risk) of metabolic diseases, especially type 2 diabetes mellitus and conditions related thereto (e.g. diabetic complications, such as micro- and/or macrovascular complications), in type 2 diabetes patients who have not been previously treated with an antihyperglycemic agent.

In a further embodiment, the pharmaceutical composition according to present invention can be used in treating and/or preventing (including slowing the progression and/or delaying the onset and/or reducing the risk) of metabolic diseases, especially type 2 diabetes mellitus and conditions related thereto (e.g. diabetic complications, such as micro- and/or macrovascular complications), in type 2 diabetes patients with insufficient glycemic control despite therapy with metformin.

In a further embodiment, the pharmaceutical composition according to present invention can be used in combination with a sulphonylurea in treating and/or preventing (including slowing the progression and/or delaying the onset and/or reducing the risk) of metabolic diseases, especially type 2 diabetes mellitus and conditions related thereto (e.g. diabetic complications, such as micro- and/or macrovascular complications), in type 2 diabetes patients with insufficient glycemic control despite therapy with metformin and a sulphonylurea.

In a further embodiment, the pharmaceutical composition according to present invention can be used in combination with an insulin (particularly basal insulin) in treating and/or preventing (including slowing the progression and/or delaying the onset and/or reducing the risk) of metabolic diseases, especially type 2 diabetes mellitus and conditions related thereto (e.g. diabetic complications, such as micro- and/or macrovascular complications), in type 2 diabetes patients with insufficient glycemic control despite therapy with metformin and an insulin (particularly basal insulin).

The pharmaceutical composition (tablet) according to the invention, wherein linagliptin is comprised in an amount of 2.5 mg and metformin hydrochloride is comprised in an amount of 750 mg or 1000 mg, is administered orally as two tablets once daily to the patients.

The pharmaceutical composition (tablet) according to the invention, wherein linagliptin is comprised in an amount of 5 mg and metformin hydrochloride is comprised in an amount of 1000 mg, is administered orally as one tablet once daily to the patients.

In an embodiment, the pharmaceutical composition (tablet) according to the invention may be administered under fed conditions to a patient in need thereof. In another embodiment, the pharmaceutical composition (tablet) according to the invention may be administered under fasted conditions to a patient in need thereof. In a further embodiment, the pharmaceutical composition (tablet) according to the invention may be administered in the evening (such as e.g. with meal) to a patient in need thereof.

The invention also relates to a pharmaceutical composition according to this invention for use in a method as described hereinbefore and hereinafter.

The invention also relates to a use of a pharmaceutical composition according to this invention for the manufacture of a medicament for use in a method as described hereinbefore and hereinafter.

### DEFINITIONS

The term “active ingredient” of a pharmaceutical composition according to the present invention means the DPP-4 inhibitor and/or metformin according to the present invention.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference ≧85 cm in men and 90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 100 mg/dL (5.6 mmol/L). The word “fasting” has the usual meaning as a medical term.

The term “hyperglycemia” is defined as the condition in which a subject has a fasting blood glucose concentration above the normal range, greater than 100 mg/dL (5.6 mmol/L). The word “fasting” has the usual meaning as a medical term.

The term “hypoglycemia” is defined as the condition in which a subject has a blood glucose concentration below the normal range, in particular below 70 mg/dL (3.89 mmol/L) or even below 60 mg/dl.

The term “postprandial hyperglycemia” is defined as the condition in which a subject has a 2 hour postprandial blood glucose or serum glucose concentration greater than 200 mg/dL (11.1 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/l), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/l.

The term “impaired glucose tolerance” or “IGT” is defined as the condition in which a subject has a 2 hour postprandial blood glucose or serum glucose concentration greater than 140 mg/dl (7.8 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.8 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Furthermore, insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl.1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):

HOMA-IR=[fasting serum insulin (μU/mL)]×[fasting plasma glucose(mmol/L)/22.5]

As a rule, other parameters are used in everyday clinical practice to assess insulin resistance. Preferably, the patient's triglyceride concentration is used, for example, as increased triglyceride levels correlate significantly with the presence of insulin resistance.

Patients with a predisposition for the development of IGT or IFG or type 2 diabetes are those having euglycemia with hyperinsulinemia and are by definition, insulin resistant. A typical patient with insulin resistance is usually overweight or obese, but this is not always the case. If insulin resistance can be detected, this is a particularly strong indication of the presence of pre-diabetes. Thus, it may be that in order to maintain glucose homoeostasis a person have e.g. 2-3 times as high endogenous insulin production as a healthy person, without this resulting in any clinical symptoms.

The methods to investigate the function of pancreatic beta-cells are similar to the above methods with regard to insulin sensitivity, hyperinsulinemia or insulin resistance: An improvement of beta-cell function can be measured for example by determining a HOMA-index for beta-cell function (Matthews et al., Diabetologia 1985, 28: 412-19), the ratio of intact proinsulin to insulin (Forst et al., Diabetes 2003, 52(Suppl.1): A459), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “pre-diabetes” is the condition wherein an individual is pre-disposed to the development of type 2 diabetes. Pre-diabetes extends the definition of impaired glucose tolerance to include individuals with a fasting blood glucose within the high normal range 100 mg/dL (J. B. Meigs, et al. Diabetes 2003; 52:1475-1484) and fasting hyperinsulinemia (elevated plasma insulin concentration). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes. Insulin resistance can be confirmed in these individuals by calculating the HOMA-IR score. For the purpose of this invention, insulin resistance is defined as the clinical condition in which an individual has a HOMA-IR score >4.0 or a HOMA-IR score above the upper limit of normal as defined for the laboratory performing the glucose and insulin assays.

The term “type 1 diabetes” is defined as the condition in which a subject has, in the presence of autoimmunity towards the pancreatic beta-cell or insulin, a fasting blood glucose or serum glucose concentration greater than 125 mg/dL (6.94 mmol/L). If a glucose tolerance test is carried out, the blood sugar level of a diabetic will be in excess of 200 mg of glucose per dL (11.1 mmol/I) of plasma 2 hours after 75 g of glucose have been taken on an empty stomach, in the presence of autoimmunity towards the pancreatic beta cell or insulin. In a glucose tolerance test 75 g of glucose are administered orally to the patient being tested after 10-12 hours of fasting and the blood sugar level is recorded immediately before taking the glucose and 1 and 2 hours after taking it. The presence of autoimmunity towards the pancreatic beta-cell may be observed by detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging). Typically a genetic predisposition is present (e.g. HLA, INS VNTR and PTPN22), but this is not always the case.

The term “type 2 diabetes” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference >40 inches or 102 cm in men, and >35 inches or 94 cm in women; or with regard to a Japanese ethnicity or Japanese patients defined as waist circumference ≧85 cm in men and ≧90 cm in women;
2. Triglycerides: ≧150 mg/dL
3. HDL-cholesterol <40 mg/dL in men
4. Blood pressure ≧130/85 mm Hg (SBP ≧130 or DBP ≧85)
5. Fasting blood glucose ≧100 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor and Diagnose”, TH-Books Verlagsgesellschaft mbH, Frankfurt/Main, 2000.

According to a commonly used definition, hypertension is diagnosed if the systolic blood pressure (SBP) exceeds a value of 140 mm Hg and diastolic blood pressure (DBP) exceeds a value of 90 mm Hg. If a patient is suffering from manifest diabetes it is currently recommended that the systolic blood pressure be reduced to a level below 130 mm Hg and the diastolic blood pressure be lowered to below 80 mm Hg.

The definitions of NODAT (new onset diabetes after transplantation) and PTMS (post-transplant metabolic syndrome) follow closely that of the American Diabetes Association diagnostic criteria for type 2 diabetes, and that of the International Diabetes Federation (IDF) and the American Heart Association/National Heart, Lung, and Blood Institute, for the metabolic syndrome. NODAT and/or PTMS are associated with an increased risk of micro- and macrovascular disease and events, graft rejection, infection, and death. A number of predictors have been identified as potential risk factors related to NODAT and/or PTMS including a higher age at transplant, male gender, the pre-transplant body mass index, pre-transplant diabetes, and immunosuppression.

The term “gestational diabetes” (diabetes of pregnancy) denotes a form of the diabetes which develops during pregnancy and usually ceases again immediately after the birth. Gestational diabetes is diagnosed by a screening test which is carried out between the 24th and 28th weeks of pregnancy. It is usually a simple test in which the blood sugar level is measured one hour after the administration of 50 g of glucose solution. If this 1 h level is above 140 mg/dl, gestational diabetes is suspected. Final confirmation may be obtained by a standard glucose tolerance test, for example with 75 g of glucose.

The term “hyperuricemia” denotes a condition of high serum total urate levels. In human blood, uric acid concentrations between 3.6 mg/dL (ca. 214 μmol/L) and 8.3 mg/dL (ca. 494 μmol/L) are considered normal by the American Medical Association. High serum total urate levels, or hyperuricemia, are often associated with several maladies. For example, high serum total urate levels can lead to a type of arthritis in the joints kown as gout. Gout is a condition created by a build up of monosodium urate or uric acid crystals on the articular cartilage of joints, tendons and surrounding tissues due to elevated concentrations of total urate levels in the blood stream. The build up of urate or uric acid on these tissues provokes an inflammatory reaction of these tissues. Saturation levels of uric acid in urine may result in kidney stone formation when the uric acid or urate crystallizes in the kidney. Additionally, high serum total urate levels are often associated with the so-called metabolic syndrome, including cardiovascular disease and hypertension.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

Other or further features and advantages of the present invention will become apparent from the following examples. The following examples serve to illustrate, by way of example, the principles of the invention without restricting it.

The following examples (as well as the features or aspects included therein) relate to particularly interesting embodiments or aspects of the present invention.

### Examples

Linagliptin/Metform in HCl extended-release fixed-dose-combination tablets are film-coated tablets manufactured using typical processes and equipment for wet-granulation, tableting and film-coating. The core tablet contains metformin HCl for extended-release and may be based on an expandable polymeric swelling formulation that increases gastric retention and extends drug release from the matrix. This metformin HCl core tablet may be film-coated (spray-coated) with up to four layers (e.g. barrier coat layer, immediate-release active coat layer, color coat layer, final coat layer), one of which (active coat layer) contains the active pharmaceutical ingredient linagliptin to add the immediate-release active component.

Tablet Coating—Barrier Coat

Purified water is charged into a stainless steel mixing vessel equipped with a suitable mixer. While mixing at a speed that ensures vortex formation, the Film Forming System is added and mixed for at least 45 minutes and until no lumps are visually present. Mixing of the coating solution is continued throughout the coating operation.

An appropriate quantity of metformin HCl extended release cores are charged to the coating pan of the perforated coating system equipped with a peristaltic pump, sceen filter, a spray bar with six nozzles and helical baffles. After the cores are preheated, pan rotation and spraying of the coating suspension begin. A fixed amount of the barrier coating suspension, which incorporates approximately 15% excess to account for losses during spraying, is applied to the cores. Upon completion of the application of the coating solution, the cores are dried at 65° C. for 10 minutes. The barrier-coated cores are then allowed to cool (outlet temperature not more than 35° C.).

Tablet Coating—Linagliptin Active Coat

Purified water is charged into a stainless steel mixing vessel equipped with a suitable mixer. While mixing at a speed that ensures vortex formation, polyethylene glycol (PEG 6000) is added, then L-arginine is added and the materials are mixed for a minimum of 20 minutes and until they are visually dispersed. Mixing continues as linagliptin is added, then talc is added and the materials are mixed for a minimum of 15 minutes and until they are visually dispersed. Mixing continues as the Film Forming System (HPMC 2910-3 and PEG 8000) is added and the materials are mixed for a minimum of 45 minutes and until no lumps are visually present. Mixing of the coating suspension is continued throughout the coating operation.

An appropriate quantity of barrier-coated tablet cores are charged to the coating pan of the perforated coating system equipped with a peristaltic pump and two spray nozzles. After the cores are preheated, pan rotation and spraying of the coating suspension begin. A fixed amount of the active coating suspension, which incorporates approximately 10% excess to account for losses during spraying, is applied. For example, the cores are coated to obtain a weight gain of 4.7%; to account for losses during spraying, an amount of coating solution equivalent to approximately 5.4% weight gain is used for spraying. Upon completion of the application of the coating solution, the cores are dried (target pan speed 3 rpm, target inlet air temperature 55° C.) for 15 minutes. The active-coated cores are then allowed to cool (product temperature not more than 35° C.).

The following compositions can be obtained.

TABLE A
Linagliptin/Metformin HCl extended release
formulation, FDC 2.5 mg/750 mg

| Component | mg/tablet |
|---|---|
| Extended release core | |
| Metformin hydrochloride (Metformin HCl) | 750 |
| Polyethylene Oxide | 355.2 |
| (e.g. high molecular weight such as about 7,000,000, such | |
| as WSR-303) | |
| Hydroxypropyl methylcellulose (Hypromellose) | 25.5 |
| (e.g. viscosity about 5 mPas for a 2% solution in water at | |
| 20° C., such as HPMC 2910-5 (Methocel ™ E5)) | |
| Magnesium Stearate | 11.3 |
| Purified water | —* |
| Barrier coat: | |
| Film Forming System: | 68.3 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 2.5 |
| Film Forming System: | 40.0 |
| Hydroxypropyl methylcellulose (e.g. HPMC of 3 cP, such | |
| as HPMC 2910-3, e.g. 95% w/w) and | |
| Polyethylene glycol (e.g. PEG 8000, e.g. 5% w/w) | |
| Arginine (e.g. L-Arginine) | 10.0 |
| Polyethylene glycol (e.g. PEG 6000) | 10.0 |
| Talc | 9.0 |
| Purified water | —* |

*Processing agent water used in manufacturing and removed by drying

TABLE B
Linagliptin/Metformin HCl extended release
formulation, FDC 2.5 mg/1000 mg

| Component | mg/tablet |
|---|---|
| Extended release core | |
| Metformin hydrochloride (Metformin HCl) | 1000 |
| Polyethylene Oxide | 369.9 |
| (e.g. high molecular weight such as about 7,000,000, such | |
| as WSR-303) | |
| Hydroxypropyl methylcellulose (Hypromellose) | 34.1 |
| (e.g. viscosity about 5 mPas for a 2% solution in water at | |
| 20° C., such as HPMC 2910-5 (Methocel ™ E5)) | |
| Magnesium Stearate | 15.0 |
| Purified water | —* |
| Barrier coat: | |
| Film Forming System: | 85.0 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 2.5 |
| Film Forming System: | 40.0 |
| Hydroxypropyl methylcellulose (e.g. HPMC of 3 cP, such | |
| as HPMC 2910-3, e.g. 95% w/w) and | |
| Polyethylene glycol (e.g. PEG 8000, e.g. 5% w/w) | |
| Arginine (e.g. L-Arginine) | 10.0 |
| Polyethylene glycol (e.g. PEG 6000) | 10.0 |
| Talc | 9.0 |
| Purified water | —* |

*Processing agent water used in manufacturing and removed by drying

TABLE C
Linagliptin/Metformin HCl extended release
formulation, FDC 5 mg/1000 mg

| Component | mg/tablet |
|---|---|
| Extended release core | |
| Metformin hydrochloride (Metformin HCl) | 1000 |
| Polyethylene Oxide | 369.9 |
| (e.g. high molecular weight such as about 7,000,000, such | |
| as WSR-303) | |
| Hydroxypropyl methylcellulose (Hypromellose) | 34.1 |
| (e.g. viscosity about 5 mPas for a 2% solution in water at | |
| 20° C., such as HPMC 2910-5 (Methocel ™ E5)) | |
| Magnesium Stearate | 15.0 |
| Purified water | —* |
| Barrier coat: | |
| Film Forming System: | 85.0 |
| Hydroxypropyl cellulose (e.g. 33.75% w/w), | |
| Hydroxypropyl methylcellulose (e.g. HPMC of 3 cP, such | |
| as Hypromellose 2910 3 cP, e.g. 33.75% w/w), | |
| Talc (e.g. 20.0% w/w) and | |
| Titanium dioxide (e.g. 12.5% w/w) | |
| Purified water | —* |
| Active (API) coat: | |
| Linagliptin (as API) | 5.0 |
| Film Forming System: | 40.0 |
| Hydroxypropyl methylcellulose (e.g. HPMC of 3 cP, such | |
| as HPMC 2910-3, e.g. 95% w/w) and | |
| Polyethylene glycol (e.g. PEG 8000, e.g. 5% w/w) | |
| Arginine (e.g. L-Arginine) | 20.0 |
| Polyethylene glycol (e.g. PEG 6000) | 10.0 |
| Talc | 9.0 |
| Purified water | —* |

*Processing agent water used in manufacturing and removed by drying

Optionally, the above compositions may be further coated by a color coat layer and/or a clear final coat. Typically, the color coat and the final coat may represent 1-4% w/w (preferably 1-2% w/w) of the total composition and each comprise one or more film-forming agents, one or more plasticizers, one or more optional glidants, and one or more optional pigments and/or colors. For example, a color coat may comprise hydroxypropyl methylcellulose (e.g. HPMC of 3 cP and/or HPMC of 6 cP), polyethylene glycol (e.g. PEG 8000), titanium dioxide and, optionally, a pigment (e.g. iron oxid yellow and/or iron oxide red), color or lake. For example, a final coat may comprise hydroxypropyl methylcellulose (e.g. HPMC of 3 cP) and polyethylene glycol (e.g. PEG 8000) as well as a polishing wax (e.g. carnauba wax).

1. A pharmaceutical composition comprising
a) an extended release core comprising metformin or metformin hydrochloride and one or more excipients;
b) a barrier coating; and
c) an immediate release coating comprising a DPP-4 inhibitor and one or more excipients;
wherein the barrier coating comprises:
one or more film-coating agents,
an optional plasticizer, and
optionally, a glidant, and/or an anti-tacking agent, and/or a pigment, and wherein the barrier coating is free of polydextrose.

2. The pharmaceutical composition according to claim 1, wherein the extended release core a) is a formulation comprising metformin hydrochloride, a swellable and/or extended release polymer, and one or more further excipients.

3. The pharmaceutical composition according to claim 1, wherein the immediate release coating c) is a film coat formulation further comprising:
one or more film-coating agents selected from the group consisting of hydroxypropyl methylcellulose (HPMC), polyvinyl alcohol (PVA), ethyl cellulose, hydroxypropyl cellulose, polydextrose, methacrylic and/or acrylic polymer, or a mixture thereof,
optionally one or more plasticizers selected from the group consisting of polyethylene glycol, propylene glycol, diethyl phthalate, tributyl sebacate and/or triacetin, or a mixture thereof,
optionally a glidant selected from the group consisting of talc, magnesium stearate and fumed silica, and
optionally one or more pigments and/or colorants.

4. The pharmaceutical composition according to claim 1, wherein the immediate release coating c) is a film coat formulation comprising linagliptin as the DPP-IV inhibitor and L-arginine as a stabilizer.

5. The pharmaceutical composition according to claim 4, wherein the weight ratio of the L-arginine to linagliptin is within the range from about 4:1 to about 2:1.

6. The pharmaceutical composition according to claim 3, wherein the one or more film-coating agents in the immediate release coating c) is hydroxypropyl methylcellulose having a nominal viscosity selected from the group consisting of 3, 5, 6, 15 and 50 cP.

7. The pharmaceutical composition according to claim 3, wherein the one or more plastizicers in the immediate release coating c) is selected from the group consisting of polyethylene glycol (PEG) 400, 3000, 4000, 6000 and 8000.

8. The pharmaceutical composition according to claim 3, wherein the plasticizer in the immediate release coating c) is propylene glycol.

9. The pharmaceutical composition according to claim 3, wherein the glidant in the immediate release coating c) is talc.

10. The pharmaceutical composition according to claim 1, wherein the immediate release coating c) comprises or consists essentially of:
Linagliptin as the DPP-IV inhibitor in an amount from 1 to 10 mg,
L-arginine,
polyethylene glycol,
hydroxypropyl methylcellulose, and
talc;
wherein
linagliptin is present in an amount 1-10% w/w,
L-arginine is present in an amount of 4-40% w/w,
the weight ratio L-arginine: linagliptin is 1:1 to 5:1,
polyethylene glycol has an average molecular weight of about 6000 and is present in an amount of 8-20%,
hydroxypropyl methylcellulose has a nominal viscosity of 3 cP and is present in an amount of 40-70% w/w,
polyethylene glycol has an average molecular weight of about 8000 and is present in an amount of 0-5% w/w, and
talc is present in an amount of 5-20% w/w,
wherein % w/w is based on the weight of the immediate release coating.

11. The pharmaceutical composition according to claim 1, wherein:
the one or more film-coating agents in the barrier coating b) is selected from the group consisting of hydroxypropyl methylcellulose and hydroxypropyl cellulose, or a mixture thereof.

12. The pharmaceutical composition according to claim 1, wherein the barrier coating b) comprises
hydroxypropyl methylcellulose having a nominal viscosity of 3 cP, and hydroxypropyl cellulose,
wherein the weight ratio of hydroxypropyl methylcellulose: hydroxypropyl cellulose is from about 10:1 to about 1:10.

13. The pharmaceutical composition according to claim 1, wherein the barrier coating b) comprises or consists essentially of:
hydroxypropyl methylcellulose, hydroxypropyl cellulose, titanium dioxide and talc, wherein
the barrier coating b) has a total weight of from 50 to 100 mg, and/or
the barrier coating b) is represent in an amount from about 5% w/w to about 15% w/w based of the weight total pharmaceutical composition.

14. The pharmaceutical composition according to claim 1, wherein the metformin hydrochloride is present in a unit dosage strength of 500, 750, 850, 1000 or 1500 mg.

15. The pharmaceutical composition according to claim 4, wherein the linagliptin is present in a unit dosage strength of 0.5, 1, 2.5 or 5 mg.

16. The pharmaceutical composition according to claim 1, which is a tablet for oral administration.

17. The tablet according to claim 16 further comprising a color coating d) and/or final coating e) over the immediate release coating c).

18. The tablet according to claim 17, wherein the color coating d) and/or final coating e) is each a film coat formulation independently comprising
one or more film-coating agents selected from the group consisting of hydroxypropyl methylcellulose (HPMC), polyvinyl alcohol (PVA), ethyl cellulose, hydroxypropyl cellulose, polydextrose, methacrylic and/or acrylic polymer, or a mixture thereof, optionally one or more plasticizers selected from the group consisting of polyethylene glycol, propylene glycol, diethyl phthalate, tributyl sebacate and/or triacetin, or a mixture thereof, optionally a glidant selected from the group consisting of talc, magnesium stearate and fumed silica, and
optionally one or more pigments and/or colorants.

19. The tablet according to claim 17, wherein the color coating d) and/or final coating e) is each a film coat formulation independently comprising a film-coating agent, a plasticizer, and, optionally, a glidant, one or more pigments and/or colors.

20. A method of using the pharmaceutical composition according to claim 1 for treating and/or preventing metabolic diseases and conditions related thereto,
either in type 2 diabetes patients who have not been previously treated with an antihyperglycemic agent,
or in type 2 diabetes patients with insufficient glycemic control despite therapy with one or two conventional antihyperglycemic agents selected from the group consisting of metformin, sulphonylureas, thiazolidinediones, glinides, alpha-glucosidase blockers, GLP-1 or GLP-1 analogues, and insulin or insulin analogues.

21. The method according to claim 20, wherein linagliptin is comprised in an amount of 2.5 mg, and metformin hydrochloride is comprised in an amount of 750 mg or 1000 mg.

22. The method according to claim 21, wherein the composition is administered as two tablets once daily to the patients.

23. The method according to claim 20, wherein linagliptin is comprised in an amount of 5 mg, and metformin hydrochloride is comprised in an amount of 1000 mg.

24. The method according to claim 23, wherein the composition is administered as one tablet once daily to the patients.

25. The pharmaceutical composition according to claim 2, wherein the swellable and/or extended release polymer comprises poly(ethylene oxide).
